(19) United States
(12) Patent Application Publication
(10) Pub. No.: US 2005/0032207 A1
(43) Pub. Date: Feb. 10, 2005

(54) Method for isolating, culturing and differentiating intestinal stem cells for therapeutic use

(76) Inventors: Anna Wobus, Gatersleben (DE); Alexandra Rolletschek, Quedlinburg (DE); Cornelia Wiese, Gatersleben (DE); Blyszczuk Przemyslaw, Gatersleben (DE); St-Onge Luc, Etthler (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/489,348

(22) PCT Filed: Sep. 12, 2002
(86) PCT No.: PCT/EP02/10248

(30) Foreign Application Priority Data
Sep. 12, 2001 (EP) 01121943.3

(51) Int. Cl. C12N 5/08

(52) U.S. Cl. 435/366; 435/371

(57) ABSTRACT

The present invention relates to methods for the isolation, culture, and production of undifferentiated somatic intestinal stem/progenitor cells of mammalian, preferably human origins. The resulting stem/progenitor cells resemble properties of embryonic stem (ES) and multipotent progenitor cells with respect to morphology, biochemical property, and in pluripotency.

### DESCRIPTION

The present invention relates to the field of the in vitro isolation and culture of undifferentiated adult stem cells and methods of producing such cells. More specifically, the present invention relates to methods and compositions for the production and genetic manipulation of stem cells from the intestine of mammals, preferably humans, the generation of specialised cells derived from such intestinal stem cells, the therapeutic use of those cells for tissue replacement, and the use of such cells in drug screening assays.

### BACKGROUND OF THE INVENTION

Stem cells are undifferentiated or immature cells that have the capacity to self renew and to give rise to various specialised cell types. Once differentiated or induced to differentiate, stem cells can be used to repair damaged and malfunctioning organs. Stem cells can be of embryonic, fetal or adult origin.

Embryonic stem cells& can be isolated from the inner cell mass of pre-implantation embryos (ES cells) or from the primordial germ cells found in the genital ridges of post-implanted embryos (EG cells), or they can be generated by nucleus transfer into enucleated oocytes and blastocyst development. When grown in special culture conditions such as spinner culture or hanging drops, both ES and EG cells aggregate to form embryoid bodies (EB). EBs are composed of various cell types similar to those present during embryogenesis. When cultured in appropriate media, EBs can be used to generate in vitro differentiated phenotypes, such as extraembryonic endoderm, hematopoietic cells, neurons and glia, cardiomyocytes, skeletal muscle, pancreatic, liver, endothelial, lipid, cartilage, bone, and vascular muscle cells.

At the molecular level, ES and EG cells express a number of genes that are highly specific for the undifferentiated status of these cells. The transcription factor Oct-4 (also called Pou5f1, Oct-3, Oct3/4) has been shown to be required for establishing and maintaining the undifferentiated phenotype of ES cells and plays a major role in determining early events in embryogenesis and cellular-differentiation (Nichols et al., 1998, Cell 95:379-391; Niwa et al., 2000, Nature Genet. 24:372-376). Oct-4 is down-regulated as stem cells differentiate into specialised cells such as hematopoietic cells, neurons, cardiomyocytes, skeletal muscle cells, pancreatic beta-cells, and vascular cells. Stage-specific embryonic antigens-1, -3, and -4 (SSEA-1, SSEA-3, SSEA-4) are glycoproteins specifically expressed in early embryonic development and are markers for ES cells (Solter and Knowles, 1978, Proc. Natl. Acad. Sci. USA 75:5565-5569; Kannagi et al., 1983, EMBO J 2:2355-2361). Elevated expression of the enzyme Alkaline Phosphatase (AP) is another marker associated with undifferentiated embryonic stem cells (Wobus et al., 1984, Exp. Cell 152:212-219; Pease et al., 1990, Dev. Biol. 141:322-352). Other stem/progenitor cells markers include the intermediate neurofilament nestin (Lendahl et al., 1990, Cell 60:585-595; Dahistrand et al., 1992, J. Cell Sci. 103:589-597), the membrane glycoprotein prominin/AC133 (Weigmann et al., 1997, Proc. Natl. Acad. USA 94:12425-12430; Corbeil et al., 1998, Blood 91 :2625-22626), the transcription factor Tcf-4 (Korinek et al, 1998, Nat. Genet. 19: 379-383; Lee et al., 1999, J. Biol. Chem. 274:1566-1572), and the transcription factor Cdx1 (Duprey et al., 1988, Genes Dev. 2:1647-1654; Subramanian et al., 1998, Differentiation 64:11-18). While the therapeutic potential of stem cells is well recognised, the use of human ES and EG cells present important ethical and social concerns related to the destruction of human embryos for the isolation and generation of these cells. The isolation and propagation of adult human stem cells with properties similar to embryonic stem cells would overcome this ethical dilemma.

Adult (somatic) stem cells with extensive self-renewal capacities have been identified in some tissues such as epidermis (Jones & Watt, Cell 73 (1993), 713-724), epithelial layer of the small intestine (Potten & Loeffler, Development 110 (1990), 1001-1020) the hematopoietic system (Cross & Enver, Curr. Opin. Genet. Dev. 7 (1997), 609-613). Neural stem cells and neurogenic activities were identified within specific regions of the adult mammalian brain (Johansson et al., Cell 96 (1999), 25-34); Gage, Science 287 (2000), 1433-1438). In other organs stem cells have a low regenerative capacity (e.g. pancreas, kidney, brain) or have not been identified (e.g. heart). However, adult stem cells are rare and often difficult to identify and isolate. For example, only an estimated 1 in 10 000 to 15 000 cells in the bone marrow is a hematopoietic stem cell (see Weissman, 2000, Cell 100:157-168). The best characterized is the hematopoietic stem cell. This is a mesoderm-derived cell that has been purified based on cell surface markers and functional characteristics. The hematopoietic stem cell, isolated from bone marrow, blood, cord blood, fetal liver and yolk sac, is the progenitor cell that reinitiates hematopoiesis for the life of a recipient and generates multiple hematopoietic lineages (see Fei, R., et al., U.S. Pat. No. 5,635,387; McGlave, et al., U.S. Pat. No. 5,460,964; Simmons, P., et al., U.S. Pat. No. 5,677,136;1 5 Tsukamoto, et al., U.S. Pat. No. 5,750,397; Schwartz, et al., U.S. Pat. No. 759,793; DiGuisto, et al., U.S. Pat. No. 5,681,599; Tsukamoto, et al., U.S. Pat. No. 5,716,827; Hill, B., et al., Exp. Hernatol. (1996) 24 (8): 936-943).

Adult stem cells are also found within the intestinal tissue of organisms. The intestinal epithelium is a highly regenerative tissue. It is composed of four differentiated cell types: enterocytes, goblet, enteroendocrine and Paneth cells. Potent stem cells found in the crypt of the villus ensures replacement of the entire cell population of the epithelium every 5-7 days (see Clatworthy and Subramanian, 2001, Mech. Develop. 101:3-9). Primary cultures of intestinal cells can be established from epithelial aggregates, however, no methods are currently available in the prior art for the establishment of primary cultures from dissociated cells (see for example, Winton in Stem Cell Biology, D. R. Marshak et al., ed, Cold Spring Harbor Laboratory Press, 2001, 550pp). Furthermore, the methods currently available in the art do not allow the establishment of conditions that allow the isolation and culture of undifferentiated intestinal stem cells capable of differentiating into cells of another tissue.

### SUMMARY OF THE INVENTION

The present invention provides methods for the isolation, culture, and production of undifferentiated somatic intestinal stem (IS) cells of mammalian, preferably human origin. IS cells may be derived from the epithelium of the duodenum, stomach, ileum, jejunum, or large intestine. In particular, this invention provides methods for the isolation, culture, and production of multipotent stem or progenitor cells of the intestinal epithelium (IE; in this invention, the term intestinal stem cells (IS) includes IE derived cells as well as of the intestinal tissue). These cells are cultured under conditions which allow long term culture of cells in an undifferentiated or progenitor-like state while retaining the potential to differentiate into a variety of cell and tissue types. The resulting IS cells resemble properties of embryonic stem (ES) cells with respect to morphology, biochemical property, and in pluripotency.

An object of the invention is to provide methods for producing human or mammal IS cell lines which present ES cells or multipotent progenitor-like properties. Another object of the invention is to provide human or mammal pluripotent IS cell lines in general, as well as differentiated cell lines derived from IS cells. A further embodiment of the invention is to provide genetically modified cells, cell lines, or tissues using the IS cells of the invention. Another embodiment of the invention is to provide IS cells or IS derived stem cells of restricted developmental lineage for transplantation or therapeutic use. Yet a further embodiment of the invention is to provide IS cells or IS derived cells for the characterisation of cellular responses to biological, chemical, or pharmacological agents in cell based drug screening assays.

### DETAILED DESCRIPTION OF THE INVENTION

Before the present methods are described, it is understood that this invention is not limited to the particular methodology, protocols, cell lines, vectors, and reagents described as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications mentioned herein are incorporated herein by reference for the purpose of describing and disclosing the cell lines, vectors, and methodologies which are reported in the publications which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the”, include plural referents unless the context clearly indicates otherwise. Thus, for example, reference to “a cell” includes one or more such cells, or a cell line derived from such cells, “a reagent” includes one or more of such different reagents, and reference to “the method” includes reference to equivalent steps and methods known to those of ordinary skill in the art that could be modified or substituted for the methods described herein.

A technical problem underlying the present invention is to provide a method for the isolation, culture, and differentiation of adult somatic stem (progenitor) cells from the intestinal epithelia. Another technical problem underlying the present invention is obtaining an undifferentiated intestinal stem cell which present ES cell-like characteristics and properties such as the expression of specific markers, such as the Oct-4 and alkaline phosphatase genes. Yet another problem is the isolation, propagation, and differentiation of nestin-positive stem cells from the intestinal epithelia or intestinal tissue. Yet another technical problem is to induce the differentiation of the intestinal stem cells into a wide variety of cell types for therapeutic use. The solution to said technical problems is achieved by the embodiments characterised in the claims.

Thus the present invention relates to methods for obtaining an intestinal stem or progenitor cell which exhibit ES cell-like characteristics and properties comprising:
(a) Isolating one or more stem cells from an intestinal epithelium of mammalian origin, preferably human
(b) Cultivating intestinal stem or progenitor cells of human or other mammal origin on feeder cells, and in culture conditions allowing growth in an undifferentiated multipotent state
(c) Optionally cultivating said IS cells, particularly IE-derived multipotent stem or progenitor cells, to form embryoid bodies or embryoid-like bodies
(d) Optionally cultivating the IS-derived embryoid bodies or embryoid-like bodies in media allowing growth of undifferentiated stem cells by the addition of factors or cells which inhibit differentiation and
(e) Optionally cultivating IS cell derived embryoid bodies or embryoid-like bodies in specific differentiation media allowing differentiation into specific cell types.

In one embodiment, the invention provides a method of producing somatic human or other mammal intestinal stem or progenitor cells which exhibit ES cell-like characteristics and properties. The starting material is human or other mammal intestinal tissue obtained by surgical techniques such as, for example but by no means limited to, pancreaticoduodenectomy (also known as the “Whipple procedure”), Roux-en-Y technique, gastric resection, gastrectomy, laparoscopic, laparoscopic Roux-en Y gastric bypass, push enteroscopy, operative endoscopy, or other surgical procedures. These methods are well described in the art; see, for example, Sivak, ed., “Gastroenterologic Endoscopy”, 2nd Edition, W.B. Saunders Co. 2000, 1611pp; Jamieson and Debas ed., “Surgery of the upper gastrointestinal tract”, Lippincott Williams and Wilkins publisher, 1994, 618pp. Furthermore, intestinal tissue of human or mammal origin can also be obtained from deceased donors. The material may be obtained from a foetus, newborn, child, or adult.

The intestinal tissue can comprise portion of the stomach, duodenum, jejunum, ileum, cecum, colon, e.g. ascending colon, transverse colon, descending colon, sigmoid colon, rectum, anal canal, and/or appendix. The smooth muscle is mechanically removed or dissected from the intestinal tissue and the remaining tissue is washed several times in a suitable buffered solution containing antibiotics. Examples of such solution in practising the present invention can be composed of Hanks' balance salt solution (Gibco/BRL) containing between 0,01 and 0,1 μg/ml, preferably 0,05 μg/ml streptomycin (Gibco/BRL); between 0,01 and 0,1 Units/ml, preferably 0,05 Units/ml penicillin (Gibco/BRL); between 10 and 100 μg/ml, preferably 50 μg/ml gentamycin (Gibco/BRL); between 1 and 100 μg/ml , preferably 2,5 μg/ml amphotericin (Gibco/BRL); between 1 and 100 mg/ml, preferably 2 mg/ml ciprobay (Bayer). Additional antibiotics, such as but not limited to vancomycin, metronidazol, and fluconazol, can also be added according to the manufacturers recommendations. Cells of the intestinal tissue are then removed by mechanical scraping of the tissue followed by dissociation in an enzymatic solution. Examples of such solutions in practising the present invention can be the digestion of the intestinal tissue for 15 to 30 minutes in a non-enzymatic dissociation solution, such as accutase (PAA Laboratories. GmbH) or for 30 to 60 minutes in a solution containing between 10 and 200 units/ml, preferably 75 units/ml collagenase type 1 (Sigma), and between 0.01 and 10 units/ml, preferably 0,02 units/ml dispase (Gibco/BRL).

After short centrifugation to pellet the cells, intestinal cells are plated and cultured in petri dishes containing suitable feeder cells, preferably human or non-human mammalian embryonic cells, such as mouse embryonic fibroblastic feeder cells previously inactivated by treatment with 100 μg/ml mitomycin C for 3 hours. Methods for preparing embryonic feeder cells are well described in the art; see, for example, Joyner, “Gene Targeting: A Practical Approach”, Oxford University Press, New York, 1993; Mansouri “Gene Targeting by Homologous Recombination in Embryonic Stem Cell”, Cell Biology: A Laboratory Handbook, second ed., Academic Press, 1998; Wobus et al., 1984, supra, Wobus et al., “In Vitro Differentiation of Embryonic Stem Cells and Analysis of Cellular Phenotypes” in: Methods in Molecular Biology: Gene Knockout Protocols, Tymms and Kola Eds, vol. 158, Humana Press Inc. 2001). Alternatively, intestinal cells can be cultured on mouse embryonic fibroblast STO cell lines which can be obtained commercially (ATCC CRL 1503; ATCC 56-X) or on human fetal feeder layer cultures (see Richards et al., Nature Biotechn online, Aug. 5, 2002).

Intestinal stem (IS) cells are preferably cultured in an appropriate cultivation medium on embryonic fibroblastic feeder cells. The term ‘cultivation medium’ refers to a suitable medium capable of supporting growth of IS cells which remain in an undifferentiated state. Examples of suitable culture media in practising the present invention are prepared with a base of Dulbecco's modified Eagle's medium (DMEM, Life Technologies) supplemented with 1 5% heat-inactivated foetal calf serum (FCS, Gibco); between 500 μM and 1 mM preferably 0.1 mM non-essential amino acids; between 500 μM and 1 mM preferably 0.1 mM beta-mercaptoethanol; between 1 mM and 10 mM preferably 2 mM glutamine; between 500 μM and 1 mM preferably 0.1 mM sodium pyruvate; 100 Units/ml penicillin; 100 μg/ml streptomycin. Instead of DMEM, also the Iscove's modification of DMEM can be used (IMDM, Life Technologies).

An effective amount of factors are then added. The term “effective amount” as used herein is the amount of such described factors as to permit a beneficial effect of the growth and viability of the IS cells using judgment common to those in the art of cell culturing and by the teachings supplied herein. Such factors can be used individually or in combination.

A first class of factors are cytokines and factors which are ligands for receptors that can heterodimerise with the signal transduction molecule termed glycoprotein 130 (gp 130). For example but not limited to, between 1 ng/ml and 1 μg, preferably 10 ng/ml leukaemia inhibitory factor (LIF; a growth factor that prevents differentiation of ES cells), or between 1 ng/ml and 1.00 μg/ml, preferably 50 ng/ml hyper-interleukin-6 (Fischer et al., 1997, Nature Biotech. 15:142-145), interleukin-6/soluble interleukin-6 receptor or other members of the IL-6 family of cytokines.

A second class of factors are those which elevate intracellular cAMP levels. For ex ample, but not limited to, between 1 μM and 100 μM, preferably 10 μM forskolin (Sigma); between 1 μM and 100 μM, preferably 10 μM cholera toxin, between 500 μM and 10 mM, preferably 0,1 mM isobutylmethylxanthine.

A third class of factors are growth factors of mammalian, e.g. human origins. For example but not limited to, between 1 ng/ml and 100 μg/ml, preferably 500 ng/ml basic fibroblast growth factor (bFGF); between 1 ng/ml and 100 μg/ml, preferably 20 ng/ml epithelial growth factor (EGF).

Intestinal stem (IS) cells including intestinal epithelial derived stern cells or progenitor precursbor cells are pluripotent cells of human or mammal origin that can be cultured in an undifferentiated pluripotent state, yet retain the ability to be differentiated into a variety of cell and tissue types. In accordance with the present invention, the terms “pluripotent” and “pluripotential cells” refer to cells which retain the developmental potential to differentiate into a wide range of cells of all three primary germ layers (endoderm, mesoderm, ectoderm) or cell lineages, such as muscle cells, cardiac cells, neuronal cells, glial cells, epithelial cells, intestinal cells, kidney cells, bone cells, cartilage cells, skin cells, pancreatic cells including islet cells and, insulin-producing beta-cells, or hepatocytes. The term “adult” and “adult stem cell” are used herein to describe cells that are found in a foetus, newborn, child or adult organism as opposed to the terms “embryonic”, and “embryonic stem cell” which are used to describe cells isolated from an embryo. In humans, an embryo is defined from the time of fertilisation until the end of the eighth week of gestation, when it becomes known as a foetus. The term “cell” or “cells” as used herein also refers to individual cells, cell lines or cultures derived from such cells.

Another embodiment of the invention is the ability of IS cells of human or mammalian origin to differentiate in vitro or in vivo into a wide variety of cell types including the ability to differentiate into embryonic and more highly differentiated cell types which can easily be tested by methods to those in the art. As shown in this invention, IE-derived stem/progenitor cells isolated from the intestine can also generate differentiated cell types from another tissue such as, for example, but not limited to the brain, spinal cord, pancreas, liver, and heart (see, for example, FIG. 2), and also lung, kidney, skeletal muscle, smooth muscle, skin, or hair. We have shown in this invention, that the IS cells are capable of expressing at least one marker for pluripotent cells. For example, multipotent nestin-positive progenitor cell populations from adult intestinal epithelium can proliferate and develop into various somatic cell types, including insulin-secreting pancreatic, neural, mesenchymal, mesodermal and hepatic cell types in vivo.

In another embodiment, IS cells stain positively for the presence of alkaline phosphatase (AP). In another embodiment, IS cells express the transcription factor Oct-4. In still another embodiment, IS cells express the intermediate neurofilament protein nestin. In still another embodiment, IS cells express specific cell surface antigens, such as SSEA-1, SSEA-3 and/or SSEA-4. In still another embodiment IS cells express prominin/AC133, Cdx1 and /or Tcf-4. Further, the cells may express any combination of the above markers.

In yet another embodiment of the invention, IS cells can be isolated and identified: by culturing IS cells on feeder layer of mouse embryonic fibroblasts to form embryoid bodies or embryoid-like bodies. The terms “embryoid bodies” or “embryoid-like bodies” refers to clumps of cellular structures that were found to be morphologically similar to “embryoid bodies” generated from ES cells. ES cell-derived embryoid bodies are cellular aggregates composed of ES cells and may contain tissues from all three of the embryonic germ layers: endoderm, mesoderm, and ectoderm. For example, IS cells can be dissociated by digesting said cells with between 1 Units/ml to 1000 Units/ml, preferably 56 Units/ml of collagenase type 1 (Sigma), and between 1 Units/ml to 1000 Units/ml, preferably 4 Units/ml dispase (Gibco/BRL) (at room temperature) for 10 to 60 minutes, and transferred to bacteriological plates containing DMEM supplemented with 15% FCS; between 500 μM and 1 mM, preferably 0.1 μM non-essential amino acids; between 500 μM and 1 mM preferably 0.1 mM beta-mercaptoethanol; between 1 mM and 10 mM, preferably 2 mM glutamine; between 500 μM and 1 mM, preferably 0.1 mM sodium pyruvate; between 10 Units/ml and 10000 Units/ml, preferably 1000 Units/ml LIF; between 1 ng and 100 μg bFGF; between 1 μM and 100 μM, preferably 10 μM forskolin (Sigma); 100 Units/ml penicillin; 100 μg/ml streptomycin. IS cells are incubated for 2 to 10 days at 37° C. with 5% CO₂.

Alternatively, embryoid bodies can be generated by a hanging drop method. Between 400-800 dissociated IS cells, preferably 600, are cultured in drops of 20 μl of Iscove modified Dulbecco's medium (IMDM, Gibco) supplemented with 20% FCS, L-glutamine, non-essential amino acids and 450 μM α-monothioglycerol placed on the lids of petri dishes filled with phosphate-buffered saline (PBS). Embryoid bodies are cultured in hanging drops for 2 to 5 days at 37° C. with 5% CO₂ and then transferred to bacteriological petri dishes (Greiner, Germany) and incubated a further 3 days in suspension culture. After 5 days, embryoid bodies are plated onto gelatin-coated 24 well plates, petri dishes or other suitable culture container and cultured for an additional 15 to 35 days at 37° C. with 5% CO₂ in a differentiation medium.

IS derived embryoid bodies can also be plated on petri dishes containing an extracellular matrix such as but not limited to 10 μg/cm² Collagen type 1 (Becton Dickinson Biosciences), 0,1% Gelatine (Fluka), 10 μg/cm² Fibronectin (Life Technologies), 14 μg/cm² Poly-L-Ornithin (Sigma), or 141.5 ng/cm² Laminin (Sigma).

Embryoid bodies can also be produced in spinner cultures. For example, dissociated IS cells can be seeded at a density of 10⁷ cell/ml in 250 ml siliconised spinner flasks (Life Technologies) containing 100 culture medium. After 24 hours, 150 ml culture medium is added to a final volume of 250 ml. Spinner flasks are stirred at 20 rpm using a stirrer system (Integra Biosciences). Such methods are well known in the art and can be scaled up for industrial production without undue experimentation see Wobus et al., “In Vitro Differentiation of Embryonic Stem Cells and Analysis of Cellular Phenotypes” in: Methods in Molecular Biology: Gene Knockout Protocols, Tymms and Kola Eds, vol. 158, Humana Press Inc. 2001, and Friend, J. R. et al.: (1999) “Formation and characterization of hepatocyte spheroids”, In: Morgan, J. R. and Yarmush, M. L. (eds.) Methods in Mol. Med., vol. 18: Tissue engineering methods and protocols, Humana Press Inc., Totowa, N.J.).

The term “differentiation medium” describes a culture medium which promotes the differentiation of IS cells into a specific cell type, such as neural or pancreatic beta cells or hepatocytes. An example of a differentiation medium favouring hepatocyte differentiation is a HBM basal medium (Clonetics Bio Whittaker) supplemented with 20% FCS, BSA-FAF, hydrocortisone, transferrin, insulin, ascorbic acid, hEGF, gentamycin, amphotericin. Another example of a medium favouring hepatocyte differentiation is an Iscove based medium supplemented with 20% FCS, 100 ng/ml alpha-FGF (Sigma), 20 ng/ml HGF, 10 ng/ml oncostatin M (Sigma), 10⁻⁷ M dexamethason (Sigma), 5 ng/ml insulin (Sigma), 5 mg/ml transferrin (Gibco/BRL), and 5 μg/ml Na-selenite (Sigma).

An example of a medium promoting differentiation of IS cells into neural progenitor cells or neurons and glia is a DMEM/F12 (1:1, Sigma) based medium supplemented with 25 μg/ml insulin, 50 μg/ml transferrin, 30 nM sodium selenite, 1 μg/ml laminin (Sigma), 20 nM progesterone (Sigma), 100 μM putrescin (Sigma), 100 units/ml penicillin, 100 g/ml streptomycin, 20 ng/ml EGF, and 10 ng/ml bFGF. Alternatively, 2% B 27 supplement (Gibco/BRL), and 10 nM nicotinamide (Sigma) can be added to the medium to generate pancreatic cells (see Lumelsky et al., 2001, Science 292:1389-1394). Another example of a medium promoting neurons and glia differentiation is based on Neurobasal medium (Gibco BRL) supplemented with 2% B 27 supplement, 10% FCS, 1 μg/ml Laminin, and 200 μM ascorbic acid (Roth) and survival promoting factors as described in the prior art, see, by Rolletschek et al., 2001, Mech. Dev. 105:93-104. Yet another example of a medium promoting differentiation of IS cells into a wide variety of cells including neurons and glia is EBM basal medium (Clonetics) supplemented with 5% FCS, hydrocortisone, human beta-FibroblastGrowth Factor (FGF), human vascular Endothelial Growth Factor (v EGF), R(3)-insulin-like growth factor I, ascorbic acid, human EGF, heparin, gentamycin, and amphotericin.

An example of a medium promoting differentiation of insulin-producing cells, and pancreatic cells is based on Iscove modified Dulbecco's medium (IMDM, Gibco BRL) supplemented with 20% FCS, 2 mM L-glutamine, 1:100 non-essential amino acids and 450 μM α-monothioglycerol (Sigma). In addition, such medium can contain between 1 ng/ml and 100 μg/ml, preferably 10 ng/ml EGF; between 1 ng/ml and 100 μg/ml, preferably 2 ng/ml basic Fibroblast Growth Factor (bFGF); between 1 nM and 1 mM, preferably 20 nM progesterone; between 10 ng/ml and 100 μg/ml, preferably 100 ng/ml Growth hormone; between 1 nM and 100 μM, preferably 5 nM follistatin (R&D); between 1 and 100 mM, preferably 2 nM activin (R&D); or nicotinamide between 10 nM-100 mM, preferably 10 mM.

An example of a medium favouring mesodermal cells, such as cardiomyocytes or skeletal muscle cells is based on Iscove's MDM (IMDM; Gibco BRL) supplemented with 20% FCS, 2 ng/ml TGF β1 (Strathmann Biotech GmbH), 50 ng/ml bFGF, 10 to 100 ng/ml BMP 2 or other BMPs (Genetics Institute), 20 ng/ml ActivinA (R&D Systems GmbH). Also chemical differentiation inducers, such as retinoic acid (see Wobus et al., 1997, J. Mol. Cell. Cardiol. 29: 1525-1539), activin, or suramine can be used to promote cardiomyocytes differentiation.

In one embodiment of the invention, IS cells can be genetically modified by using an expression vector comprising a polynucleotide encompassing a polypeptide-coding region, e.g. a cDNA or a gene which is placed under the control of a regulatory region allowing the initiation of transcription and introduced into IS cells by transfection methods such as electroporation, lipofection, calcium phosphate mediated, DEAE dextrans, and the like. Such methods and system are well described in the art (see, for example, Joyner, “Gene Targeting: A Practical Approach”, Oxford University Press, New York, 1993; Mansouri “Gene Targeting by Homologous Recombination in Embryonic Stem Cell”, Cell Biology: A Laboratory Handbook, second ed., Academic Press, 1998). The term “polynucleotide” means any piece of genomic DNA, cDNA, or RNA sequence that is transcribed into DNA. The polynucleotide of interest is operably linked to a regulatory element, such as a transcriptional or translational regulatory element. Regulatory elements include for example an promoter, an initiation codons, a stop codon, a mRNA stability regulatory element, and a polyadenylation signal. Expression of a polynucleotide can be assured by (i) constitutive promoters such as the. Cytomegalovirus (CMV) promoter/enhancer region, (ii) tissue specific promoters such as the insulin promoter (see, Soria et al., 2000, Diabetes 49:157), SOX2 gene promotor for neural precursor cells (see Li et al., 1998, Curr. Biol. 8:971-4), Msi-1 promotor for neuronal cells (see Sakakibara et al., 1997, J. Neuroscience 17:8300-8312), alpha-cardia myosin heavy chain promotor or human atrial natriuretic factor promotor for cardiomycte selection (Klug et al., 1996, J. clin. Invest 98:216-24; Wu et al., 1989, J. Biol. Chem. 264:6472-79), or (iii) inducible promoters such as the tetracycline inducible system.

In another embodiment, the nucleic acid sequence of interest can be inserted into a vector, such as an expression vector (see Sambrook et al., supra). Expression vectors can also contain a selection agent or marker gene that confers antibiotic resistance such as the neomycin, hygromycin, or puromycin resistance genes. The selection would include contacting the mixed differentiated cellular population with an antibiotic to which cells of the desired cell line have been conferred resistance by the expression of said line-specific gene, thereby eliminating cell of other cell lines, but not cells of the desired cell line expressing the specific gene.

Expression vectors can also contain a gene that promotes the differentiation of IS cells into a specific cell type or a nucleic acid (gene) that confers special properties to the IS cells. For example, a pancreatic gene can be introduced into IS cells to promote the differentiation of such cells into insulin-producing cells. The term “pancreatic gene” means a gene or its protein product that is involved and required for pancreas development, more preferably beta-cell differentiation. Specific non-limiting examples of such genes are Pdx1, Pax-4, Pax-6, neurogenin 3 (ngn3), Nkx6.1, Nkx6.2, Nkx2.2, HB9, BETA2/NeuroD, IsI1, HNF1-alpha, HNF1-beta, and HNF3 of human or animal origin. The polypeptide encoded by such gene can be from the same species as the stem or progenitor cells (homologous) or can be from a different species (heterologous). Each gene can be used individually or in combination. Introduction of genes encoding transcriptional regulators (like Pdx1, Pax-4, Pax-6, ngn3, or Nkx2.2) can provide more efficient proliferation and differentiation of the progenitor cell and can increase survival of such cells during in vitro cultures or after transplantation of the cell in vivo.

Suitable gene expression vectors are well known in the art; see Sambrook et al., “Molecular Cloning, A Laboratory Manual” third ed., CSH Press, Cold Spring Harbor, 2000; Gossen and Bujard, 1992, Proc. Natl. Acad. Sci. USA 89:5547-5551. These include, but are not limited to, non-viral vectors or such as plasmid or cosmid DNA expression vectors and viral expression vectors (e.g., baculovirus, adenovirus, adeno-associated virus, lentiverus, retrovirus vectors).

Cells are cultured in vitro and an exogenous gene encoding the heterologous nucleic acid is introduced into the cells by transfection or other methods. The transfected cell can then be studied in vitro or administered to a patient or other subject.

Specific constructs of interest include anti-sense molecules, which will block gene expression of desired proteins, or constructs for the expression of dominant negative mutations. A detectable marker, such as for example lac-Z, may be introduced, where upregulation of expression of a desired gene will result in an easily detected change in phenotype.

In a further embodiment, protein products of a desired gene can be delivered directly to IS cells. For example, protein delivery can be achieved by polycationic liposomes (see, Sells et al., 1995, Biotechniques 19:72-76), Tat-mediated protein transduction (see, Fawell et al., 1993, Proc. Natl. Acad. Sci. USA 91:664-668) and by fusing a protein to the cell permeable motif derived from the PreS2-domain of the hepatitis-B virus (see, Oess and Hildt (2000) Gene Ther. 7:750-758). Preparation, production and purification of such proteins from bacteria, yeast or eukaryotic cells are well known by persons skilled in the art.

Another embodiment of the invention is a method of treating a disease wherein the stem or progenitor cells or differentiated cells or a composition containing these cells are administered to a patient. The differentiated cell types include various somatic cell types, preferably pancreatic (including insulin-producing cells) neuronal, mesenchymal, mesodermal and hepatic cell types. The diseases can be, for example, but not limited to, diabetes, neuronal diseases, liver diseases, or cardiac diseases.

An additional embodiment of the invention provides a method of treating the above mentioned diseases or a diseases by transplantation therapy. In this embodiment, a method of repairing damaged tissue or missing cells in a human subject by introducing differentiated IS cells in said subject is provided. After the cells are differentiated according to the culturing method described in this invention, cells or islets are suspended in a pharmacologically acceptable carrier, for example, cell culture medium phosphate buffered saline, Krebs-Ringer buffer, or Hanks balanced salt solution with or without glucose. Differentiated IS cells may be introduced into the body of a subject by localised injection or by systemic injection. An example of such a solution in practising the present invention is the use of IS cells differentiated into insulin-producing cells for autologous treatment of diabetes. The volume of cell suspension administered to a subject will vary depending on several paramenters including the size of the subject, the severity of the disease, and the site of implantation and amount of cells in solution.

Typically the amount of cells administered to a subject will be a therapeutically effective amount. For example, between 3,000 and 100,000 equivalent surviving IS cell-derived insulin-producing cells per kilogram body weight are introduced into a diabetic patient to have a beneficial effect.

The insulin-producing cells differentiated from IS cells can be administered by any method known to one skilled in the art. For example, the cells are administered by sub-cutaneous injection or intraportally via a percutaneous transhepatic approach using local anaesthesia. Such surgical techniques are well known in the art and can be applied without any undue experimentation, see, for example, Pyzdrowski et al, 1992, New England J. Medicine 327:220-226; Hering et al., 1993, Transplantation Proc. 26:570-571; Shapiro et al., 2000, New England J. Medicine 343:230-238. Furthermore, cells are administered by implantation under the kidney capsule through the portal vein of the liver or into the spleen. Cells can also administered directly to a subject. In other embodiments, the cells are encapsulated prior to administration, such as by co-incubation with a biocompatible matrix known in the art. Several encapsulation technologies have been described in the prior art, see for example, Lanza et al., 1996, Nature Biotech 14:1107-1111, Lacy et al., 1991, Science 254:1782-84, Sullivan et al., 1991, Science 252:718-712. Nucleic acid sequences can be introduced to lower the probability of rejection of a transplanted tissue. For example, the immunogenicity of cells may be suppressed by deleting genes, that produce proteins that are recognized as foreign by the host, or by introducing genes which produce proteins, such as native host proteins, that are recognized as self proteins by the host immune system.

Other examples are the use of IS cell differentiated cardiomyocytes to treat cardiac diseases including among others but not limited to myocarditis, cardiomyopathy, heart failure, damage caused by heart attacks, atherosclerosis, and heart Valve dysfunction (see Orlic et al., 2001, Nature 410: 701-705).

Other examples are the use of IS cell differentiated liver cells to treat liver diseases including among others but not limited to acute and chronic liver failure, liver diseases (e.g., such as, for example, caused by alcohol; viral infection, metabolic disorders, drugs, or inborn error of metabolism (e.g. Hemorhomatosis, A. Wilson)), liver fibrosis, liver cirrhosis, autoimmune disease, diseases of the bile ducts and gall bladder, preferably by the above mentioned transplantation therapy.

IS cells differentiated into neuronal cells can be used to treat a disease with a neural deficit or degeneration including among others but not limited to stroke, Alzheimer's disease, Parkinson's disease (see Freed et al., 2001, N.Engl. J. Med. 344: 710-719), Huntington's disease, AIDS associated dementia, spinal cord injury, metabolic diseases effecting the brain or other nerves, see, Gage, Cell Therapy. Nature 392: 18-24 (1998), and Kirschstein and Skirboll, “Stem cell: scientific progress and future research directions”, Report prepared by the NIH, 2001.

Yet another embodiment of the invention is the use of IS cells or their differentiated derivative to study early events in human development, e.g., for the identification of genetic, molecular, and cellular events leading to developmental problems and identifying methods for preventing them. Such differentiated cells can also be used to study the effects of chromosomal abnormalities in early development, including human childhood tumors.

Yet another embodiment of the invention is the use of IS cells or their differentiated derivative to characterise cellular responses to biologic, chemical, or pharmacological agents, e.g. in cell based drug screening assays and/or toxicology studies. An “agent” refers to a chemical compound or composition capable of inducing a desired therapeutic or. prophylactic effect when properly administered to a subject or cell. For example, an agent describes a molecule, e.g. protein or pharmaceutical, with the capacity of altering or mimicking the physiological function of the IS derived differentiated calls of this invention. For example, the present invention allows the generation of IS cells for the identification and characterisation of compounds which stimulate beta-cell differentiation, insulin secretion and/or glucose response. The compound of interest is added to differentiated and undifferentiated insulin-producing cells which are grown in appropriate culture system, for example 96 and 384 well plates. A parameter is assayed to determine if the agent affects the pancreatic insulin-producing cells. For example, the secretion and/or expression of a pancreatic endocrine hormone such as insulin, glucagons, somatostatin and/or pancreatic polypeptide can be analyzed. Hormone, e.g. insulin levels in treated cells can be quantified by suitable methods, e.g. Enzyme Linked Immunoabsorbent Assay (ELISA) or Radio Immuno Assay(RIA). Using this method, a large number of compounds can be screened and compounds that induce beta-cell differentiation and increase hormone (for example, insulin) secretion can be identified readily.

If differentiated IS cells, e.g. pancreatic cells are transfected with nucleic acid constructs encoding a reporter gene (see above), an increase or decrease in the expression of the reporter gene can be analyzed using methods that detect levels or status of protein or mRNA present in the corresponding cell or detect biological activities of the reporter gene. Suitable reporter molecules or labels, which may be used, include radionuclides, enzymes, fluorescent, chemiluminescent or chromogenic agents as well as substrates, co-factors, inhibitors, magnetic particles, and the like.

IS cells differentiated e.g. into pancreatic cells can also be transfected with nucleic acid constructs encoding target genes aimed at determining biological responses of agents on effects mediated by this proteins indicating their degree of activation either during beta-cell differentiation or in fully differentiated cells. Target genes can for example be cell surface receptors, for which the effects of agents on binding of their specific ligands, agonists or antagonists or their respective downstream signalling, e.g. increase in cellular cAMP level or transcriptional activation of reporter genes is monitored. Target genes can also include intracellular proteins like protein kinases, phosphatases or nuclear hormone receptors, or everything else that is suitable for drug discovery approaches, as is state of the art in non-stem cell based cellular screening assays.

Designing such drug screening assays are well known in the art; see Harvey ed., ‘Advances in drug discovery techniques’, John Wiley and Sons, 1998; Vogel and Vogel eds., ‘Drug discovery and evaluation: Pharmaceutical assays’, Springer-Verlag Berlin, 1997).

In another embodiment, the IS or differentiated cells can be used to test candidate therapeutic drugs. For example, drug screening tests in animal models, in vitro tests using animal cells, or in vivo tests involving toxicology tests in animals may be carried out. An in vitro model can be used for screening libraries of compounds in any of a variety of drug screening techniques. Of particular interest are screening assays for agents that have a low toxicity for mammalian cells. Cultures of mammalian, preferably human, IS cells can be employed in preclinical tests. If those cells can be differentiated into specific cell types, as described in this invention, important for drug screening, those IS-derived cells are more likely to mimic the in vivo repsonse of cells/tissues and offer safer models for drug screening.

In another embodyment of the invention, mammalian, preferably human IS cells can be employed for screening potential toxins. Toxins often show different effects on different animal species, which makes it critical to employ the best in vitro model for evaluating the effects of those toxins in human cells. For example, human IS cells differentiated into liver cells can be used in evaluating drug detoxifying capabilities and represent a system for monitoring adverse effects.

Candidate agents encompass numerous chemical classes, though typically they are organic molecules, preferably small organic compounds having a molecular weight of more than 50 and less than about 2,500 Daltons. Candidate agents comprise functional groups necessary for structural interaction with proteins, particularly hydrogen bonding, and typically include at least an amine, carbonyl, hydroxyl or carboxyl group, preferably at least two of the functional chemical groups. The candidate agents often comprise carbocyclic or heterocyclic structures and/or aromatic or polyaromatic structures substituted with one or more of the above functional groups.

Candidate agents are also found among biomolecules including peptides, saccharides, fatty acids, steroids, purines, pyrimidines, nucleic acids and derivatives, structural analogs or combinations thereof. Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

### BRIEF DESCRIPTION OF THE DRAWINGS

FIG. 1 shows the general scheme for the isolation and cultivation of IS cells from mouse or rat intestinal epithelium (IE-derived stem/progenitor cells) followed by their subsequent-aggregation into embryoid bodies. Cytokines and growth factors were used in the following combinations: (I) LIF; (II) LIF+H-IL6; (III) H-IL6+bFGF (10 ng/ml), +EGF; (IV) bFGF+EGF; (V) LIF+H-IL6+bFGF+EGF.

FIG. 2 illustrates a general scheme for the differentiation of IE-derived stem/progenitor cells embryoid bodies into various specific cell types..

Medium HCM (Clonetics Bio Whittaker): (1): HBM basal media+20% FCS+BSA-FAF+hydrocortisone+transferrin+insulin+ascorbic acid+hEGF+gentamycin/amphotericin;

Medium HM: Iscove's MDM (Gibco BRL)+Additives+20% FCS+alpha-FGF (100 ng/ml, Sigma)+HGF (20 ng/ml)+Oncostatin M (10 ng/ml, Sigma)+Dexamethason (10⁻⁷ M, Sigma)+insulin (5 ng/ml, Sigma)+transferrin (5 mg/ml, Gibco BRL)+Na-selenite (5˜μg/ml, Sigma);

Medium B2: DMEM/F12 (1:1, Sigma)+insulin (25 μg/ml)+transferrin (50 μg/ml)+sodium selenite (30 nM)+laminin (1 82 g/ml, Sigma)+progesterone (20 nM, Sigma)+putrescin (100 μM, Sigma)+penicillin (100 units/ml), streptomycin (100 pg/ml)+EGF (20 μg/ml)+bFGF (10 ng/ml);

Medium EGM: 2MV (Clonetics): EBM basal media+5% FCS+hydrocortisone+hrbFGF+hvasc. EGF+R(3)-insulin-like growth factor I+ascorbic acid+hEGF+heparin+gentamycin+amphotericin;

Medium CM: Iscove's MDM (IDMDM; Gibco BRL)+add.+20% FCS+TGF β1 (2 ng/ml, Strathmann Biotech GmbH)+bFGF (50 ng/ml)+BMP 2 (10 ng/ml, Genetics Institute)+Activin A (20 ng/ml, R&D Systems);

Medium N2: medium B2+2% B 27 supplement, or medium N2+nicotinamide (10 mM, Sigma); Medium BDM: Iscove's MDM+additives+20% FCS+progesterone (20 nM)+growth hormone (100 ng/ml, ICN)+bFGF (2 ng/ml)+EGF (10 ng/ml). Abbreviation, ECM, extracellular matrix.

FIG. 3 shows the colony morphology of rat IS cell colony growing on a feeder layer of Mitomycin C-inactivated mouse fibroblasts.

FIG. 4 shows AP-positive IS-cell colonies established from rat intestinal epithelium. IS-cells were cultured on feeder cells of mitomycin C-inactivated mouse embryonic fibroblasts in DMEM with additives, 15% FCS, supplemented by (A) LIF (10 ng/ml) and H-IL-6 (B) H-IL-6 and (C) without additional cytokines. Cells and clusters were subcultured every 2 to 5 days and AP staining was performed at day 28. Bar=50 μm

FIG. 5 shows the quantitative evaluation of AP-positive colonies in rat intestinal cells cultured on mouse embryonic feeder cells. Rat intestinal epithelium was cultivated on feeder cells in DMEM with 15% FCS supplemented by additives (I), additionally LIF (10 ng/ml, II); LIF, H-IL-6-(III); H-IL-6 (IV); H-IL-6, bFGF (10 ng/ml), EGF (20 ng/ml, V); bFGF, EGF (VI); LIF, H-IL-6, bFGF, EGF (VII). Feeder layer cultivated in DMEM with 15% FCS and additives as well as feeder layer cultivated in medium supplemented by LIF and H-IL-6 were used as controls. AP-positive clusters were estimated as percentage of all colonies counted grown on feeder layer.

FIG. 6 shows the expression of the embryonic stem cell-specific transcription factor Oct-4 in primary intestinal epithelium (IE), IS-derived colonies grown on feeder cells in the presence of different cytokines/growth factors, and mouse ES cells. Oct-4 mRNA levels were analysed by RT-PCR of primary rat intestinal epithelium and IS cell-derived selected colonies propagated on feeder layer with DMEM, supplemented with 15% FCS (I); additionally with LIF (10 ng/ml, II); LIF and H-IL-6 (III); H-IL-6+bFGF (10 ng/ml); EGF (20 ng/ml, IV); bFGF+EGF (V) and LIF, H-IL-6, bFGF, EGF (VI) at day 9. Mouse ES cells of line R1 were used as positive control, feeder layer (FL), feeder layer cultivated in DMEM containing LIF and H-IL-6 as well as tridistilled water (H₂O) were used as negative controls. HPRT was used as internal standard. MW, molecular weight marker.

FIG. 7 shows the expression of embryonic cell surface marker SSEA-1 in primary cultures of rat IS cells. (A) Representative cells cultured on feeder cells in the presence of LIF. After fixation with methanol:acetone (7:3)., cells were labelled by antibody MC-480 (1:10 dilution; Developmental Studies Hybridoma Bank) recognising the SSEA-1 epitope, (B) phase contrast. (C) High magnification of SSEA-1-positive cells by confocal laser-scanning microscopy (CLSM 410, Zeiss) and (D) Differential interference contrast. Bar=10 μm

FIG. 8 demonstrates the generation and morphology of multipotent EB-derived lines established from rat intestinal epithelium. (A) Morphology of a rat IS-derived colony growing on feeder cells with DMEM, additives, 15% FCS, H-IL-6 at day 14 after 2 times passaging with trypsin (0.05%): EDTA (0.04%) 1:1. (B, C) EBs were generated by dissociation of rat IS-derived colonies with collagenase (112 U/ml) : dispase(4 U/mi, 1:1) and cultivated in DMEM supplemented by additives, 15% FCS, LIF (10 ng/ml), forskolin (10 μM; Sigma) and bFGF (10 ng/ml) within five days in suspension culture. (D) Cellular morphology of EB-derived outgrowth (further line 5) cultivated in Iscove's modification of DMEM (IMDM, Gibco BRL), 20% FCS, TGFbeta1 (2 ng/ml; Strathmann Biotech GmbH) and bFGF (50 ng/ml) three days after EB dissociation with collagenase (112 U/ml): dispase (4 U/ml, 1:1) and plating of EBs on gelatine (0.1%)-coated petri dishes. (E) Cellular morphology of EB-derived line 5 at passage 11 and (F) morphology of line 30 cultivated in EGM2MV medium supplemented by 5% FCS and heparin (all from Clonetics, Verviers, Belgium) on collagen-coated petri dishes at passage 10. Bar=50 μm

FIG. 9 shows the immunocytochemical analysis of rat IS cell embryoid body-derived cultures. Antibody epitopes are: (A) nestin, (B) GFAP, (C) oligodendrocytes specific protein, (D) synaptophysin, (E) peripherin, (F) desmin.

FIG. 10 shows the immunocytochemical analysis of rat IS cell embryoid body-derived culture in a medium favouring differentiation into insulin-producing cells. Top row, bright field; bottom row, staining with an antibody specific for insulin.

FIG. 11 shows AP-positive IS-cell colonies established from human intestinal epithelium.

FIG. 12 shows the quantitative evaluation of AP-positive colonies of cultivated human intestinal epithelium on feeder layer. (A) Human IS cells at passage 4 cultured for 13 days. (B) Human IS cells at passage 7 cultured for 25 days. Percentage of AP positive stained area for cells cultivated in different media. For each variant 20 images were analysed by the LUCIA imaging system.

FIG. 13 illustrates the expression of the germline-specific transcription factor Oct-4 and the extracellular matrix molecule TRA-1-60 in human intestinal epithelial derived progenitor stem cells. A. RT-PCR analysis of Oct-4 expression in primary intestinal epithelium and in IS cells cultured in the presence of Hyper-IL6 and/or LIF. Mouse R1 ES cells are used as positive control. Oct-4 is not expressed in the primary intestinal epithelium. However, expression can be detected in the IS cells isolated from the epithelium. B. Bright field image of human IS cells. C. Staining of human IS cells with TRA-1-60.

FIG. 14 shows the karyotype of rat epithelical derived stem/progenitor cells derived cell lines. (A) Line 5, (passage 8). (B) Line 30 (passage 9).

FIG. 15 shows the immunohistochemical analysis of mouse Rosa26-derived intestinal epithelium for Cdx1 (A) and nestin. (B) expression. Inserts (in B) show the distinct staining of nestin-positive cells within the transit amplifying region of the intestinal epithelium. Bar=50 μm (A,B) and 25 μm (inserts).

FIG. 16 shows general strategies of selective cultivation and generation of multipotent nestin-positive precursor cells from intestinal epithelium (IE).

FIG. 16A shows two principle strategies: Stage 1 cells were generated by selective-cultivation on embryonic feeder layer (FL) for 8 to 14 passages, Stage 2 and Stage 3 cells were derived by selective cultivation of IE-derived clusters (Stage 2) and IE-derived embryoid body (EB)-like aggregates (Stage 3). The microscopical pictures demonstrate the morphology of a ROSA26 mouse IE-derived colony grown for 6 days on FL in DMEM supplemented by LIF (A), and a beta-galactosidase stained cluster grown 4 days on FL (B). After cultivation for 9 days on FL in the presence of LIF and growth factors, EB-like aggregates were transferred into suspension culture for 5 days (C, D), and IE-EB-derived cells growing on different extracellular matrix (ECM) proteins (E, F: collagen I) in different cultivation media supplemented by different growth factors (GF) (medium I: G, medium II:. H). Both, from stage 1 and from stage 3 IE-derived cells, differentiated progenies of different lineages were generated. Bar=50 μm.

FIG. 16B shows the cultivation strategies of IE-derived stem/progenitor cells in different media on specific ECM matrices to differentiates various cell types.

(1) Medium HCM/HM (HCM from Clonetics Bio Whittaker): HBM basal media+20% FCS+BSA-FAF+hydrocortisone+transferrin+insulin+ascorbic acid+hEGF+gentamycin/amphotericin; IMDM (Gibco BRL)+Additives+alpha-FGF (100 ng/ml, Sigma)+HGF (20 ng/ml)+Oncostatin M (10 ng/ml, Sigma)+Dexamethason (10⁻⁷ M, Sigma)+insulin (5 ng/ml, Sigma)+transferrin (5 mg/ml, Gibco BRL)+Na-selenite (5˜μg/ml, Sigma)+20% FCS;

(2) Medium B2 (DMEM/F12 (1:1, Sigma)+insulin (25_g/ml)+transferrin (50 μg/ml)+sodium selenite (30 nM)+laminin (1 μg/ml, Sigma)+progesterone (20 nM, Sigma)+putrescin (100 μM, Sigma)+penicillin (100 units/ml), streptomycin (100 μg/ml)+EGF (20 ng/ml)+bFGF (10 ng/ml)) plus 5% FCS; Neurobasal plus B27 plus 15% FCS;

Medium EGM 2MV (Clonetics): EBM basal media+5% FCS+hydrocortisone+hrbFGF+hvasc. EGF+R(3)-insulin-like growth factor I+ascorbic acid+hEGF+heparin+gentamycin+amphotericin

(3) Medium IMDM plus MTG plus LIF plus TGFbeta plus beta FGF: Iscove's MDM (IDMDM; Gibco BRL)+add. +20% FCS+TGF β1 (2 ng/ml, Strathmann Biotech GmbH)+bFGF (50 ng/ml)+BMP 2 (10 ng/ml, Genetics Institute)+Activin A (20 ng/ml, R&D Systems);

(4) Medium B2+2% B 27 supplement, or medium N2+nicotinamide (10 mM, Sigma); Medium BbM: Iscove's MDM+additives+20% FCS+progesterone (20 nM)+growth hormone (100 ng/ml, ICN)+bFGF (2 ng/ml)+EGF (10 ng/ml).

Abbreviation, ECM, extracellular matrix.

FIG. 17 shows an immunofluorescence and confocal microscopical analysis of IE-derived clusters (Stage 2). Cells within and around IE-derived clusters were labelled by nestin (green) and Hoechst 33342 (blue) to visualise cell nuclei. Nestin-positive cells were observed within and around the clusters and shown in 9 (A) and 14 (B) days old cultures of IE-derived cells on feeder layer. Nestin-positive cells (C) were localised in ALP-positive clusters (D). An increase is observed in the number of nestin-positive cells during cultivation from day 9 to day 14 (E). LIF and LIF+bFGF+EGF, respectively, increased the number of nestin-positive cells in comparison to cells cultured only on FL in basic medium (DMEM). In contrast, the number of ALP-positive clusters is not influenced by LIF and growth factor treatment (F). No ALP-positive clusters were detected in FL cells cultured without IE cells (G). Bar=30 μm.

FIG. 18 shows an immunofluorescence analysis and confocal microscopy of IE-EB-derived cells. Shown are cells of the outgrowths of EB-like aggregates 2 days after plating, with Cdx1 (A, including Hoechst 33342-labelling of nuclei)-, nestin (B)- and GFAP (C)-positive cells. Signals after Cdx1-labelling were detected mainly in the cytoplasm (A). D shows Nomarski microscopy of Hoechst-labelled nuclei of GFAP-positive cells (C). Bar=30 μm.

FIG. 19 shows an immunofluorescence analysis of IE-derived cells differentiated into the hepatic and pancreatic lineages. Cells expressing hepatic (A-D) and pancreatic proteins (E-H) were analysed after 14 days of differentiation in specific differentiation media. Hepatocyte-like cells were labelled by nestin (A), albumin (B), alpha-1-antitrypsin (C) and cytokeratin 18 (D), pancreatic cells were labelled by insulin (E), glucagon (F) and double-stained by insulin (red) and glucagon (green), and Hoechst 33342 to visualize cell nuclei (blue) after confocal microscopy. Bars=40 μm (A-H), inserts (B-G)=10 μm.

FIG. 20 shows an immunofluorescence analysis of IE-EB-derived cells after selective differentiation into the neural and mesodermal/mesenchymal lineage. Cells expressed nestin (A), GFAP (B), Oligodendrocyte-specific protein (C), beta-III tubulin (D), desmin (E) and vimentin (E). Nestin-positive cells coexpressed also desmin (not shown here). Hoechst 33342 labelling was used to visualize nuclei (blue). Bar=40 μm.

FIG. 21 shows an immunofluorescence studies of primary cultures of IE-derived cells (day 2-5). Cells were stained with the antibody against SSEA-1 (red), co-stained with prominin, nestin, cdx (green), respectively and Hoechst 33342 to visualise cell nuclei (blue), and observed by confocal microscopy and phase contrast (B,E,I, L O). Small round cells were characterised by SSEA-1 expression and high nuclear-cytoplasmic ratio (H, insert E, see I,L,O). Mouse ES R1 cells were also SSEA-1 positive (A). C, F represent negative controls for SSEA-1 staining. SSEA-1 positive cells co-expressed prominin (G, H, I), but not cdx1 (J, K, L) and nestin (M, N, O). Bar 10 μm

FIG. 22 shows an immunofluorescence analysis and confocal microscopy of IE-derived cells. Shown are cells (of passage 14) after differentiation (14d) in B2 medium supplemented with B27, niacinamid (NA), 5%FCS, LIF, bFGF. Prominin-positive cells (A,E) were double-stained by nestin (B, F), and counterstained with Hoechst 33342 to visualize cell nuclei (C,G). D, H show phase contrast pictures. Bar=20 μm.

Tables 1 2, and 3 give examples on the quantitative evaluation of generating differentiated cells

Table 1 shows the quantitative evaluation of immunolabeling of IE-EB-derived aggregates (9-days primary culture on FL, 5 days EB cultivation in suspension) analysed 2 days after plating in DMEM+bFGF+LIF+forskolin (stage. 2)

Table 2 shows the quantitative evaluation of immunolabeling of IE-derived cell lines after selective differentiation into neural, pancreatic and hepatic cells (stage 1)

Table 3 shows the quantitative evaluation of immunolabeling of Rosa26 mouse IE-EB-derived cell lines (stage 3) after selective differentiation into neural, pancreatic, hepatic and mesenchymal cells

Table 4 shows the intracellular and secreted insulin levels in IE-derived cells after selective differentiation into the pancreatic lineage (cultivation for 14 days in B2 medium supplemented with B27, NA, 5% FCS) (Stage 3)

### EXAMPLES

A better understanding of the present invention and of its many advantages will be had from the following examples, given by way of illustration.

### Example 1

Isolation of IS cells

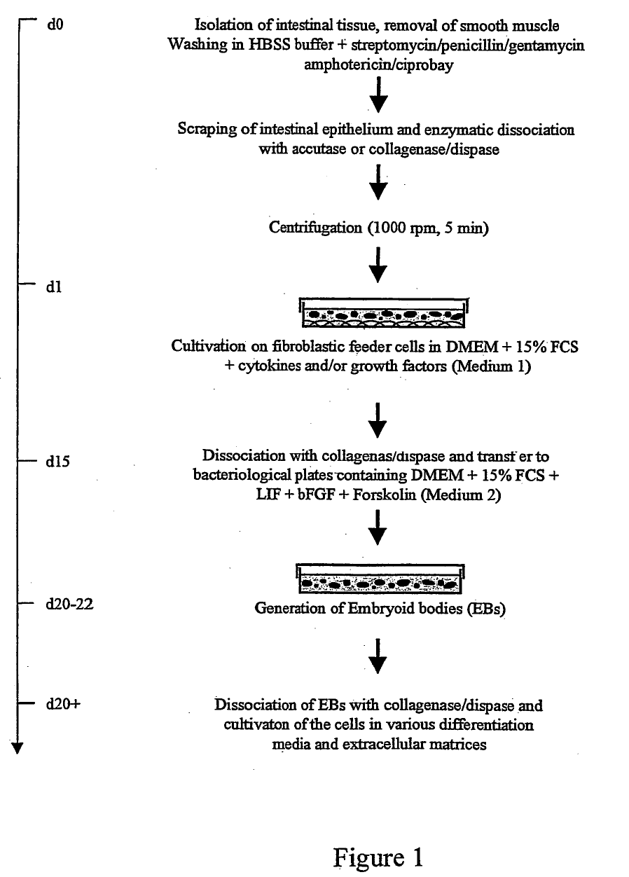

As mentioned above, in a preferred embodiment intestinal stem (IS) cells are harvested from the intestinal epithelium of human or mammalian origin (FIG. 1). For human IS cells, approximately 1 to 10 cm of small intestine was obtained during pancreaticoduodenectomy. The rat is used as model for obtaining IS cells of mammalian origin. Accordingly, the small intestine was isolated by dissection from F-344 inbred rats (Harlan Olac; age: 15-24 weeks). After flushing of lumenal contents with Hanks' balance salt solution (HBSS; Gibco BRL) containing 0.05 U/ml penicillin, 0.05 μg/ml streptomycin, 50 μg/ml gentamycin (Gibco/BRL), 2,5 μg/ml amphotericin (Gibco/BRL), 2 mg/ml ciprobay (Bayer), the smooth muscles surrounding the intestinal epithelium was mechanically removed with a scalpel, and the tissue was cut longitudinally and the pieces were washed up to 10 times in HBSS. The intestinal epithelium was then isolated by scraping and enzymatic dissociation with accutase (PAA Laboratories). For cultivation, the isolated intestinal epithelial cells were plated on Mitomycin C (50 μl/ml; Serva) inactivated mouse embryonic fibroblasts in a DMEM (Gibco BRL) based medium supplemented with 15% FCS (Biochrom KG) and containing the following cytokines and/or growth factors: 0.05 μg/ml LIF (for preparation see Fässler et al., 1996); 50 ng Hyper-IL-6 (H-IL6, used as supernatant from a hybridoma cell line see Fischer et al., 1997, Nature Biotech. 15:142-145); 10 or 500 ng/ml bFGF (Strathmann Biotech GmbH); 20 ng/ml EGF (Strathmann Biotech GmbH). Cytokines and growth factors were used in the following combinations: (I) LIF; (II) LIF+H-IL6; (III) H-IL6+bFGF (10 ng/ml), +EGF; (IV) bFGF+EGF; (V) LIF+H-IL6+bFGF+EGF. Cells were cultivated on feeder cells for eight days, subcultured 2 times and replated at day 9 into bacteriological petri dishes. Small IS cell clusters or colonies of cells could be obtained under such conditions. These IS colonies possessed a morphology similar to embryonic stem (ES) cells (FIG. 3). IS-cells were tightly compacted and the cytoplasm contained a high density of granules.

### Example 2

Pluripotency of Human and Rat IS Cells Determined by Alkaline Phosphatase (AP) Activity

As previously described, markers for pluripotent cells are often useful to identify stem cells in cultures. IS cells of rat (FIG. 4) or human (FIG. 11) origin typically manifest AP activity and AP positive cells are typically pluripotent. AP activity has been demonstrated in ES and ES-like cells in the mouse (Wobus et al., 1984, Exp. Cell 152:212-219; Pease et al., 1990, Dev. Biol. 141:322-352), rat (Ouhibi et al., 1995, Mol. Repro. Dev. 40:311-324; Vassilieva et al., 2000, Exp. Cell Res. 258:361-373), pig (Talbot et al., 1993, Mol..Repro. Dev. 36:139-137), cow (Talbot et al., 1995, Mol. Repro. Dev. 42:35-52), and in humans (Thomson et al., 1998, Science 282:1145-1147; Shamblott et al., 1998, Proc. Nat. Acad. Sci. USA 95:13726-13731; Pera et al., 2000, J. Cell Science 113:5-10). AP activity was determined by fixing human or rat IS cells in 4% paraformaldehyde at room temperature for 20 minutes followed by three washing in TM-buffer (1M maleic acid, 3.6 g Tris ad 1L H₂O pH 9.0). Cells were then stained with 0.1% Fast Red TR salt, 0.04% naphtol AA-MX phosphat resolved in TM-buffer. 25 ml staining solution includes 200 μl of a 10% MgCl₂ solution. Samples were washed with PBS.

Quantitative evaluation of AP-positive colonies in rat and human intestinal cells was also determined when different culture media was used. For rat intestinal cultures, the addition of various cytokines and growth factors slightly improved the amount of AP colonies present in the cultures (FIG. 5). However in human cultures (FIG. 12a, b), members of the IL-6 family of cytokines such as LIF and Hyper-IL6 significantly increase the amount of AP-positive IS colonies. AP-positive clusters were estimated as percentage of all colonies counted grown on feeder layer.

### Example 3

Pluripotency of Human and Rat IS Cells Determined by Oct-4 Expression, SSEA-1 Expression, or TRA-1-60 Expression.

Pluripotency of IS cells can also be studied by Oct-4 expression. The transcription factor Oct-4 has been shown to be required for establishing and maintaining the undifferentiated phenotype of ES cells, and plays a major role in determining early events in embryogenesis and cellular differentiation (Pesce et al., 1998, Bioessays 20:722-732). Oct-4 has been shown to be expressed in mouse (Nichols et al., 1998, Cell 95:379-391), rat (Vassilieva et al., 2000, Exp. Cell Res. 258:361-373) and human (Reubinoff et al., 2000, Nature Biotechnol. 18:399-404) ES or EG cells. Oct-4 is also expressed in rat IS cells (FIG. 6), and in human IS cells (FIG. 13a).

Analysis of Oct-4 gene expression in rat IS cells colonies after cultivation with different variants of cytokines and growth factors at day 9 was performed using a semiquantitative reverse transcription-polymerase chain reaction (RT-PCR) method; Single colonies were collected in lysis buffer and mRNA was isolated using Dynalbeads mRNA DIRECT micro kit (Dynal). RT-PCR amplification was performed according to the manufacturer's instructions. The RT reaction (20 μl) was performed using MuLV reverse transcriptase (Perkin-Elmer). cDNA was amplified with specific primer sequences of mouse Oct-4 and hypoxanthine guanine phosphoribosyltransferase (HPRT) as internal standard. The oligonucleotide sequences are given in parentheses in the order antisense-, sense-primer followed by the length of the amplified fragments: Oct-4 (SEQ ID No. 1: 5′-GGC GTT CTC TTT GGA AAG GTG TTC-3′ and SEQ ID No. 2: 5′-CTC GAA CCA CAT CCT TCT CT-3′; 313 bp ); HPRT (SEQ ID No. 3: 5′-GCC TGT ATC CAA CAC TTC G-3′ and SEQ ID No. 4: 5′ -GCG TCG TGA TTA GCG ATG-3′; 507 bp). Amplification was done with 15 μl from each RT reaction using following conditions: 40 seconds denaturation at 95° C., 40 seconds annealing at 61° C. and 40 seconds elongation at 72° C.; 40 cycles. Mouse R1 cells were used as positive and feeder layer of mouse embryonic fibroblasts (FL) as well as feeder cells cultivated with LIF and H-IL-6 as negative controls. Ultrapure water was always included as a control. The RT-PCR products were electrophoretically analysed on 2% agarose gels. Gels were illuminated with UV light and the ethidium bromide fluorescence signals were stored using an E.A.S.Y system (Herolab GmbH). Expression of Oct-4 in human cells was also performed as described above using oligonucleotides specific for the human Oct-4 gene: sense SEQ ID No. 5: 5′-TGA AGC AGA AGA GGA TCA CC-3′; antisense SEQ ID No. 6: 5′-CCG CAG CTT ACA CAT GTT CT-3′.

The pluripotency or rat IS cells was also evaluated by the expression of the stage-specific embryonic antigens-1 (SSEA-1; FIG. 7) which is a glycoprotein specifically expressed in early embryonic development and are markers for ES cells (Solter and Knowles, 1978, Proc. Natl. Acad. Sci. USA 75:5565-5569; Kannagi et al., 1983, EMBO J 2:2355-2361). Indirect immunofluorescence analysis was carried out on rat IS cells cultivated on feeder layer for four days and fixed with methanol:aceton (7:3) solution at −20° C. for 5 minutes. After washing (3×) with PBS, all preparations were incubated with 10% goat serum in PBS for 30 min followed by incubation with the primary antibody specific for SSEA-1 (dilution of 1:10; Developmental Studies Hybridoma. Bank.) at 37° C. in a humidified chamber for 1 hour. After washing (3×) with PBS, cells were incubated for 1 hour at 37° C. with appropriate fluorescence-labelled secondary antibodies (Jackson Immuno Research), rinsed (3×) with PBS and with A. tridest. (1×). After being embedded in Vectashield mounting medium (Vector, USA), specimens were analysed with the fluorescence microscope (Nikon, Germany) or the confocal laser scanning microscope (CLSM 410, Carl Zeiss, Jena, Germany).

The pluripotency of human IS cells was also evaluated by the expression of the extracellular matrix molecule TRA-1-60 (FIG. 13b, c) which is a specific marker for embryonic stem cells (Andrews, et al., 1984, Hybridoma 3: 347-361).

### Example 4

Rat IS Cells can be Induced to Generate Various Differentiated Cell Types.

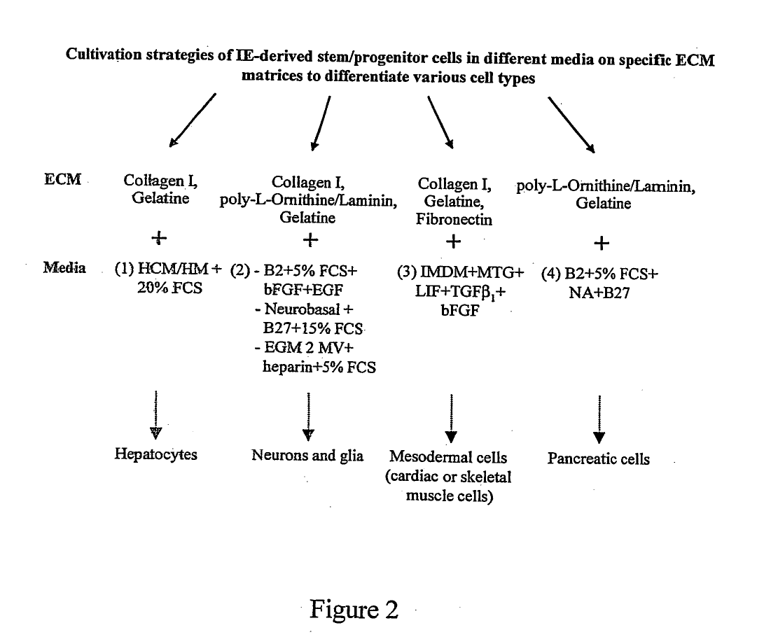

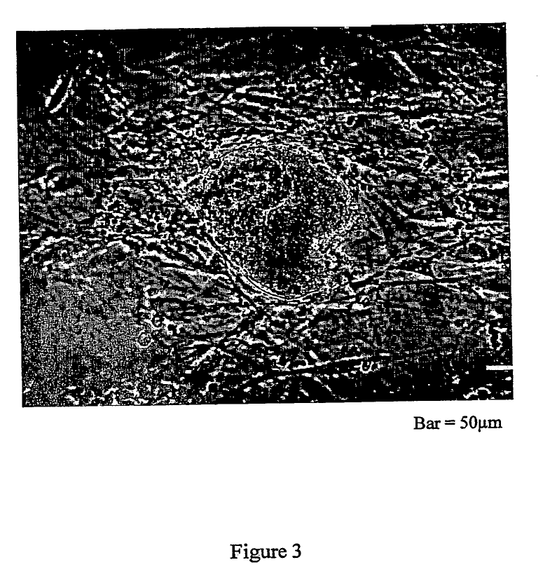

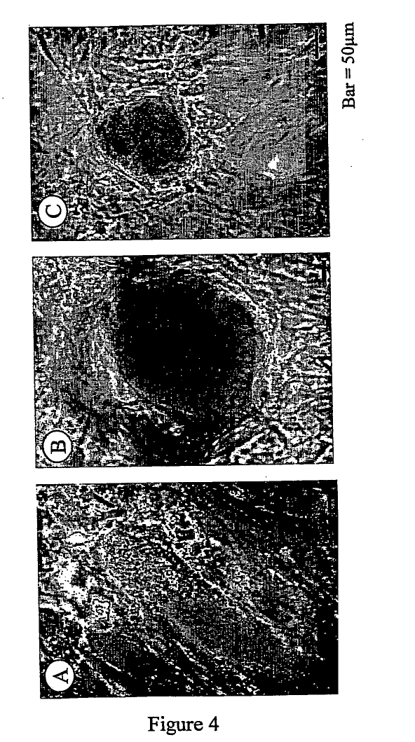

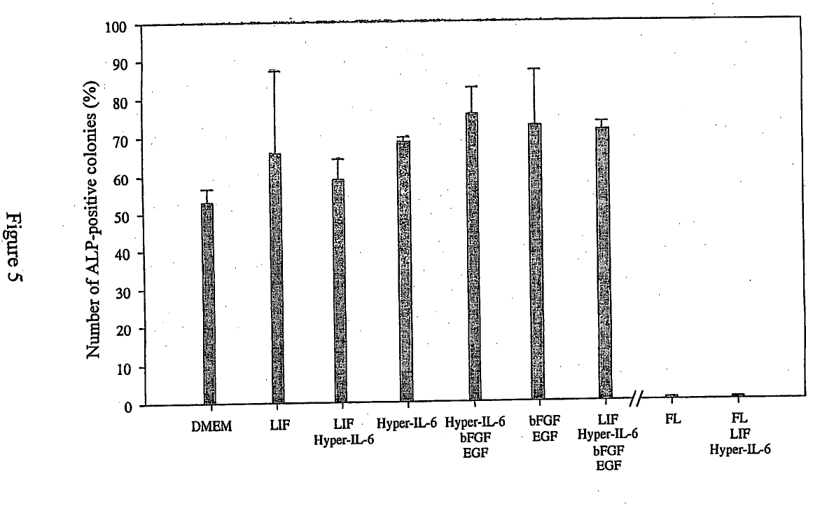

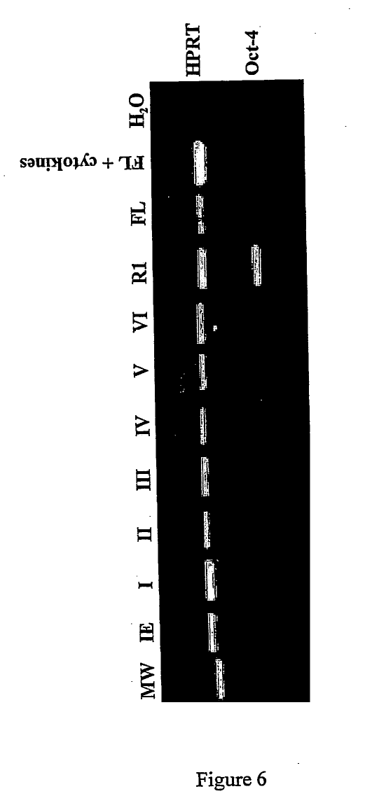

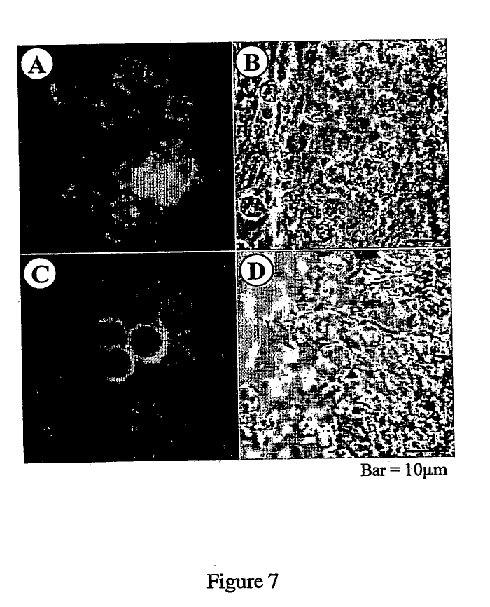

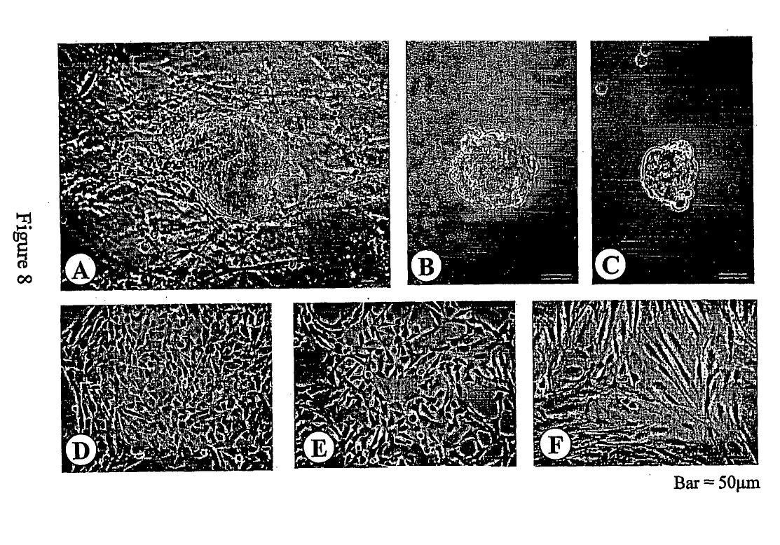

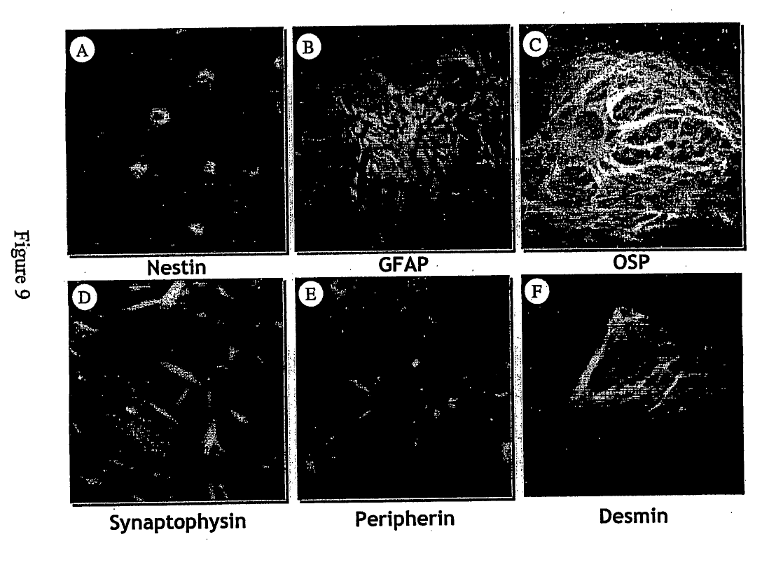

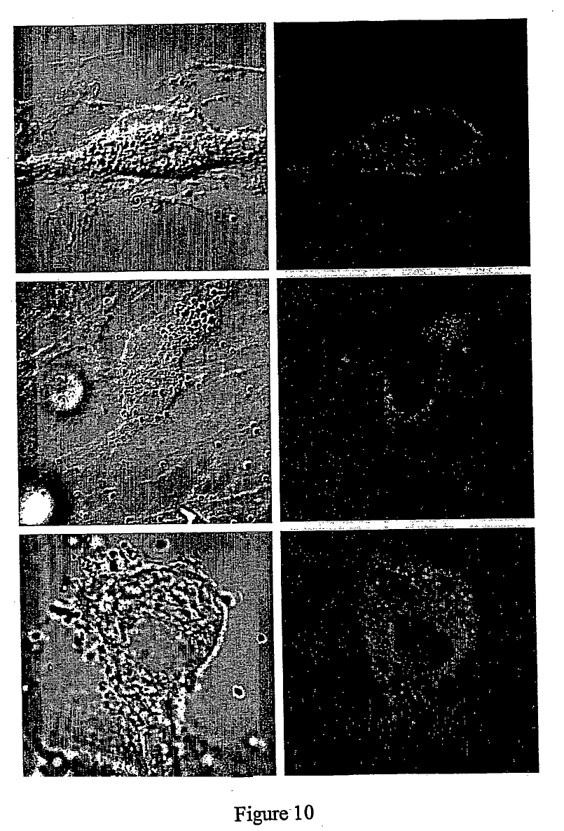

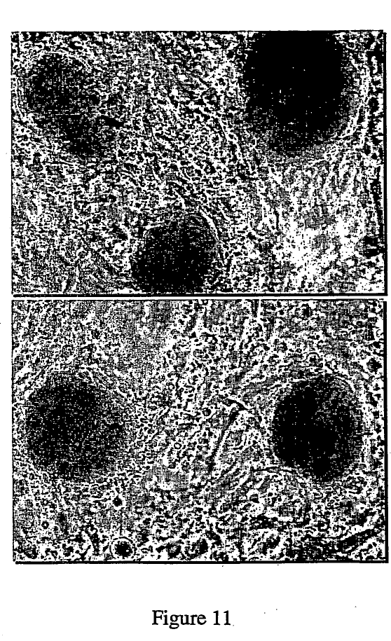

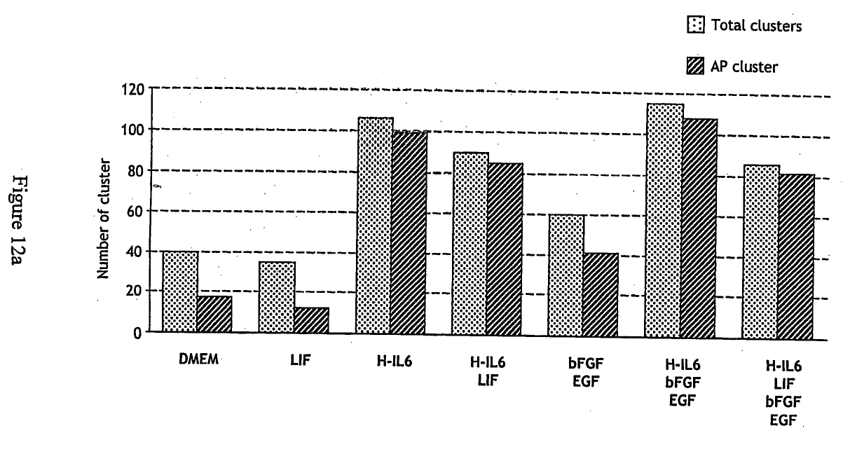

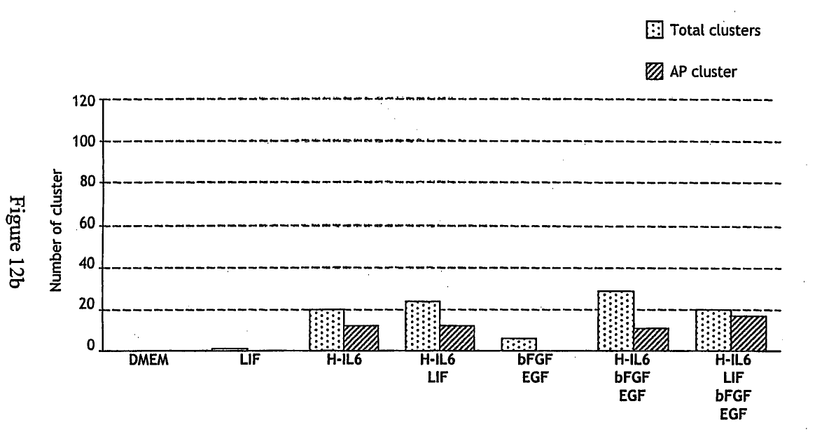

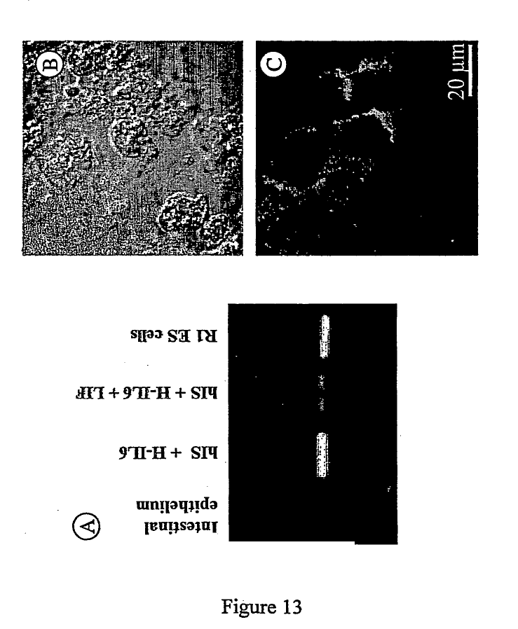

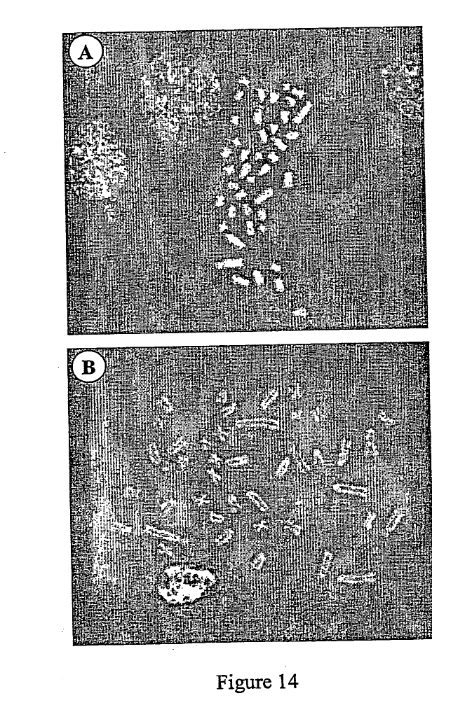

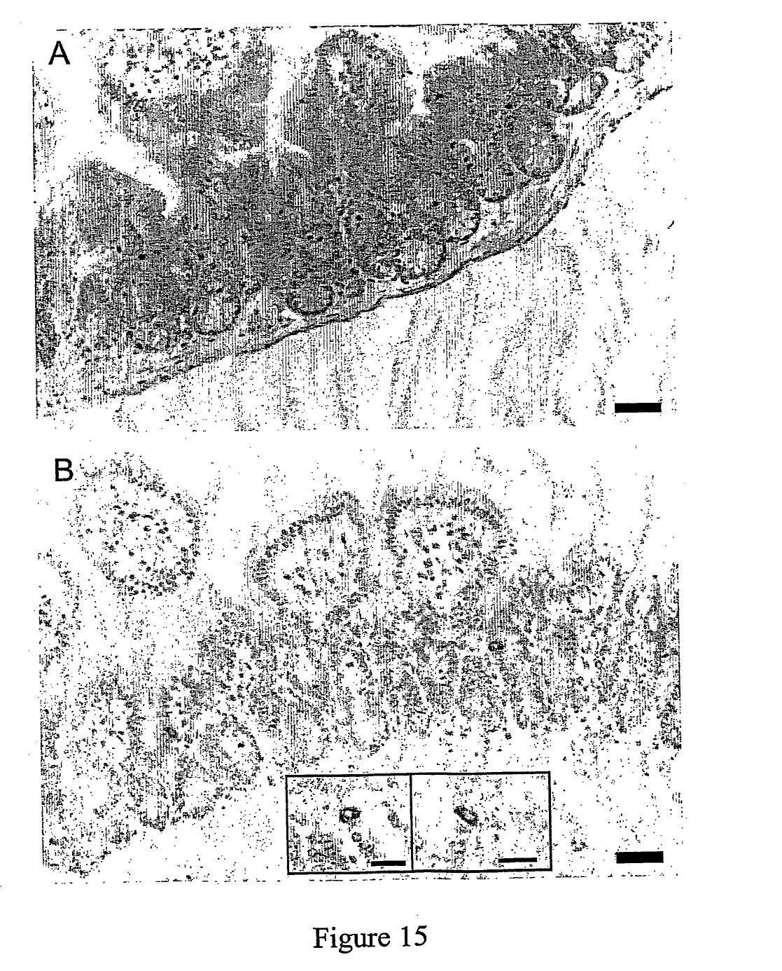

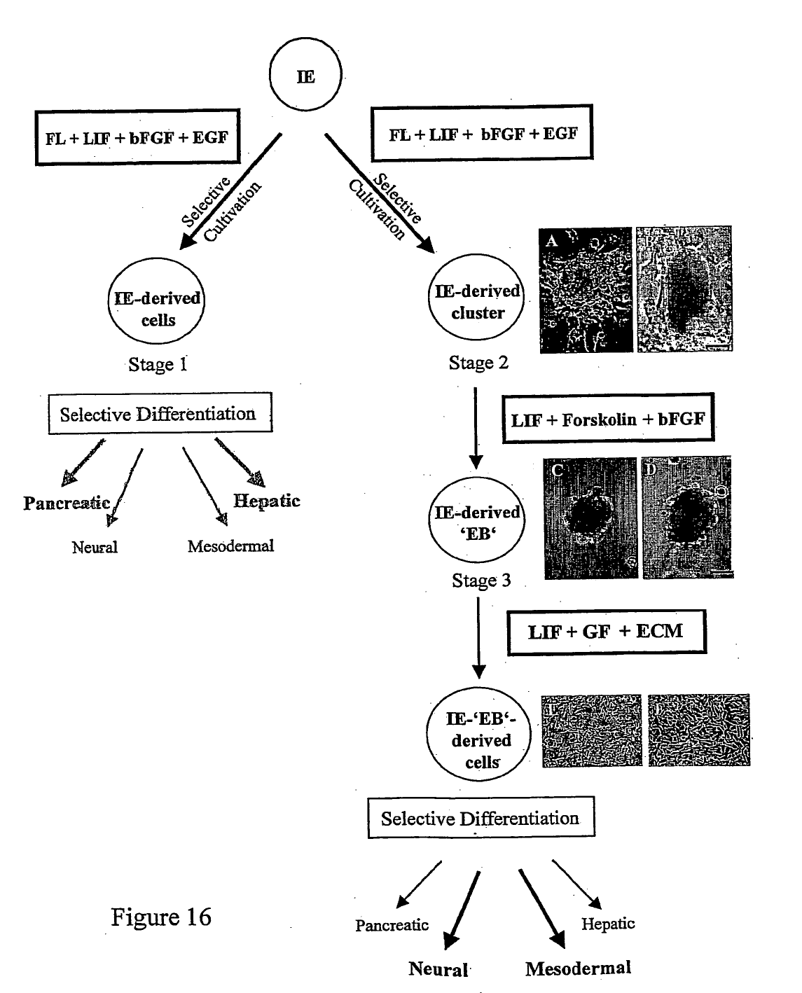

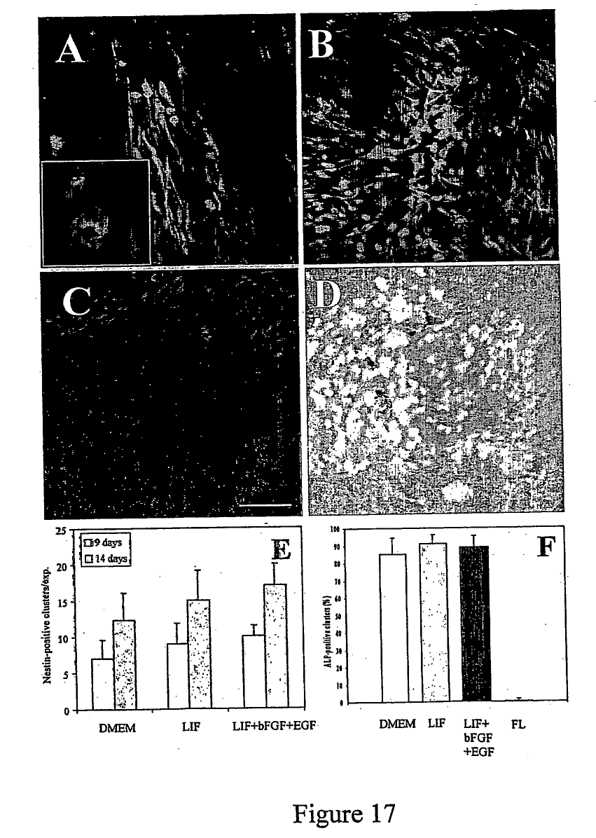

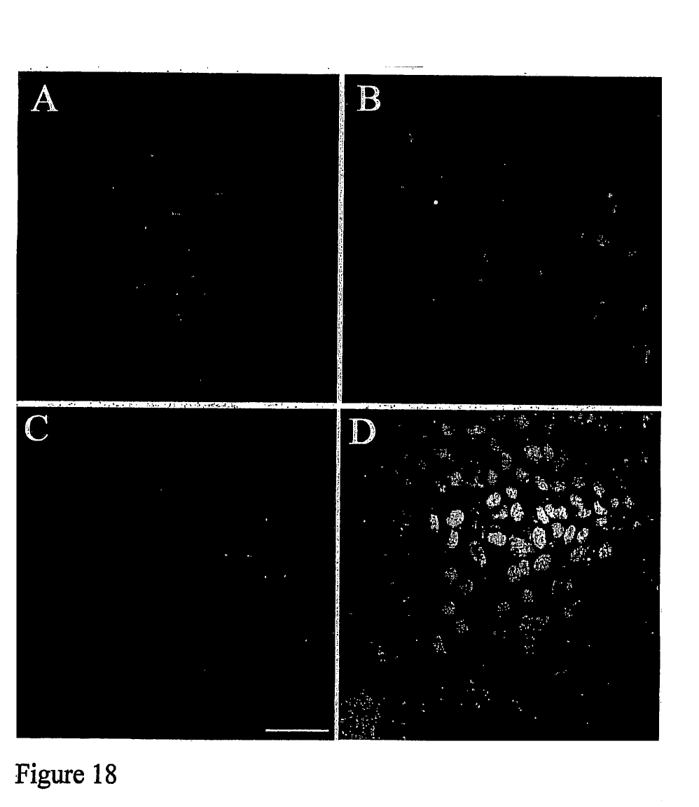

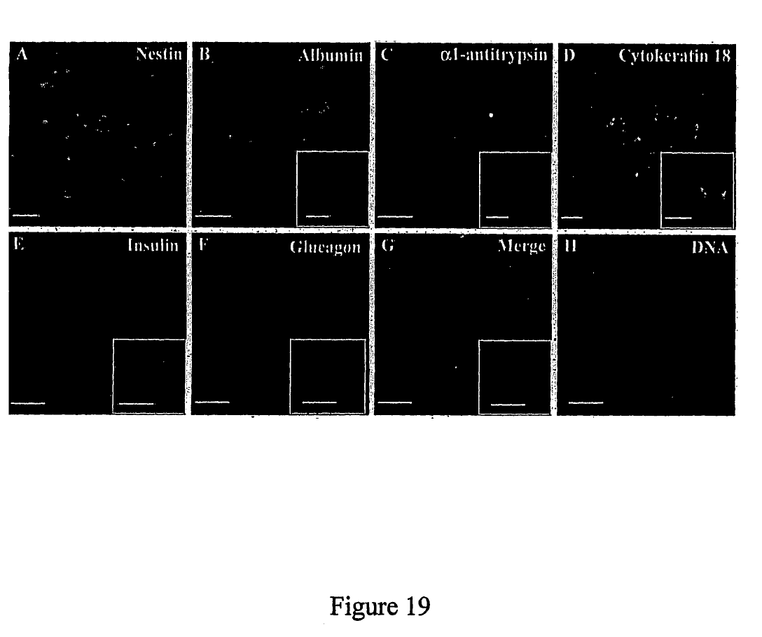

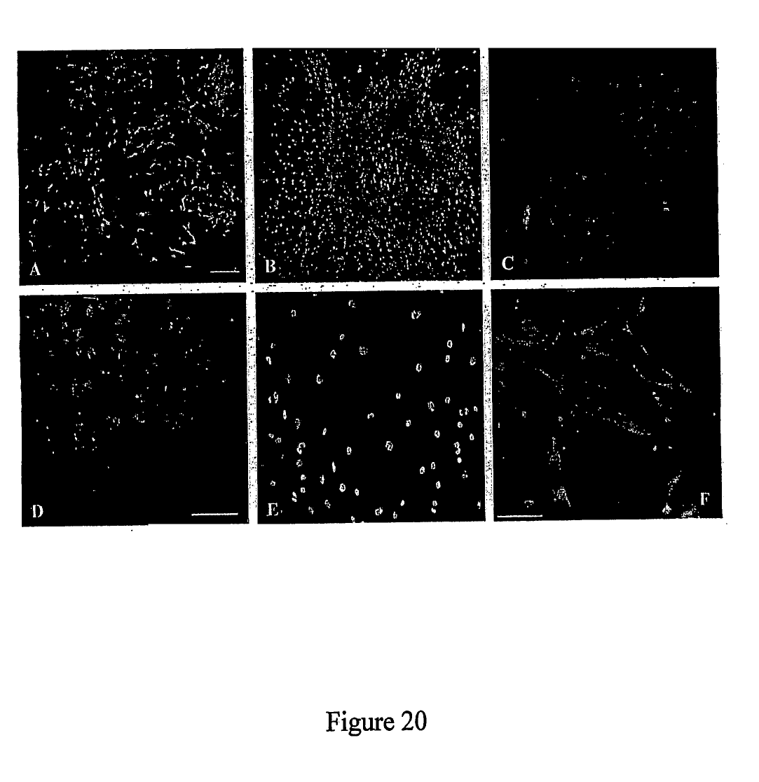

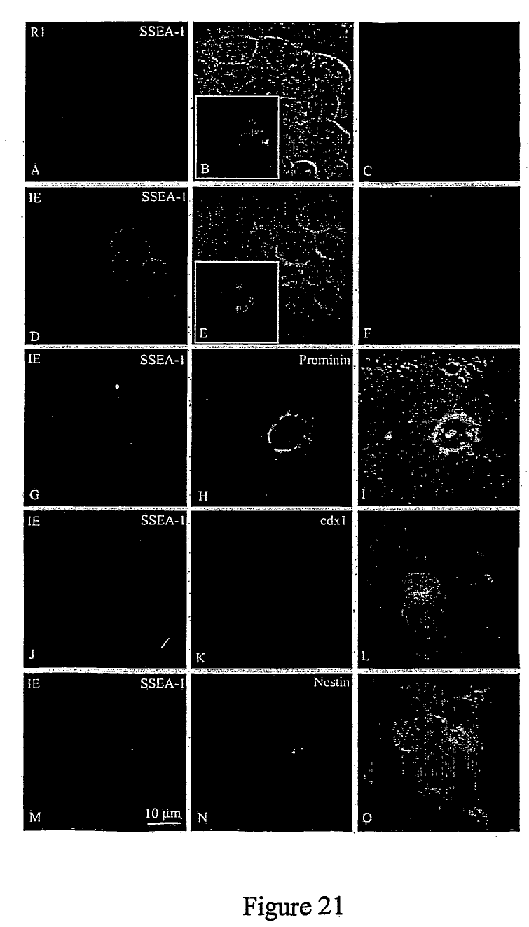

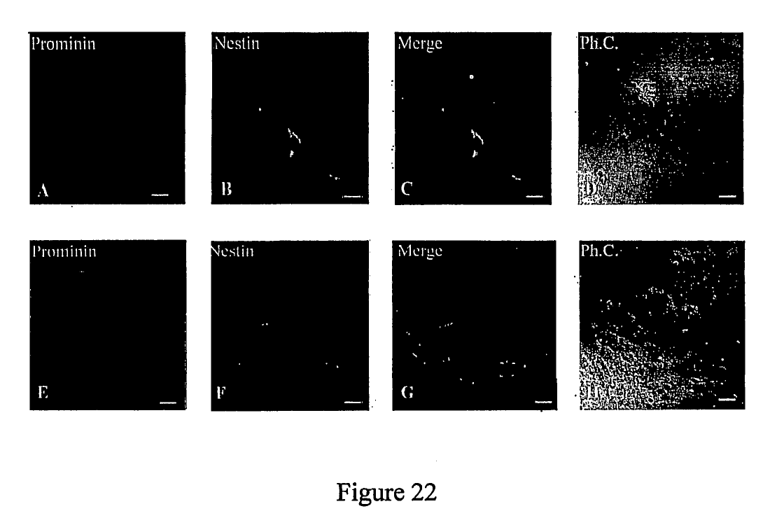

IS cells can form embryoid bodies, or embryoid-like bodies (FIG. 8b, c), and cultured in a differentiation medium to generate specific cells types, such as but not limited to, hepatocytes, neurons, glia, cardiac cells, and pancreatic cells, preferably insulin-producing cells (FIG. 2). Embryoid bodies were generated by dissociation of rat IS-derived colonies with collagenase (112 U/ml): dispase (4 U/ml, 1:1) and cultivated in DMEM supplemented by additives, 15% FCS, 10 ng/ml LIF, 10 μM forskolin (Sigma) and 10 ng/ml bFGF within five days in suspension culture. After three days of culture, embryoid bodies were dissociated with collagenase (112 U/ml):dispase (4 U/ml, 1:1) and plated on collagen-coated (10 μg/cm²) petri dishes. In one example, IS cell derived embryoid bodies were cultured in the differentiation medium EGM 2MV (Clonetics) composed of EBM basal media, 5% FCS, hydrocortisone, hrbFGF, hvasc. EGF, R(3)-insulin-like growth factor I, ascorbic acid, hEGF, heparin, gentamycin, amphotericin. Under such conditions, neural progenitor cells, pancreatic progenitor cells, a strocytes, oligodendrocytes, neurons, peripheral neurons, and muscle cells could be observed by immunohystological analysis (FIG. 9). The presence of neural progenitor, and pancreatic progenitor cells (FIG. 9a) can be detected with the expression of the intermediary filament structural protein nestin (see Lumelsky et al., 2001, Science 292:1389-1394). Astrocyte-like cells can be detected by the expression of the glial fibrillary acidic protein (GFAP; FIG. 9b). Oligodendrocytes-like cells are characterised by the expression of oligodendrocyte specific protein (OSP; FIG. 9c). Neurons and peripheral neurons, are detected by the expression of synaptic vesicle protein synaptophysin (FIG. 9d), and peripherin (FIG. 9e). Muscle cells, such as skeletal muscle cells, and cardiac cells (FIG. 9f) can be detected by the expression of desmin (see Wobus et al., “In Vitro Differentiation of Embryonic Stem Cells and Analysis of Cellular Phenotypes” in: Methods in Molecular Biology: Gene Knockout Protocols, Tymms and Kola Eds, vol. 158, Humana Press Inc. 2001).

Indirect immunofluorescence analysis was carried out as described above. The following antibodies were used in the analysis: Nestin (1:2 dilution), synaptophysin (1:250 dilution; Calbiochem), oligodendrocytes specific protein (1:20 dilution; Chemicon), peripherin (1:200 dilution; Chemicon), GFAP (1:20 dilution; Chemicon), desmin (1:4 dilution; Roche Molecular Biochemicals), respectively.

### Example 5

Rat IS Cells can be Induced to Differentiate into Insulin-producing Cells.

After formation of embryoid bodies or embryoid-like bodies, rat IS cells can be induced to differentiate into insulin-producing cells (FIG. 10) by culturing the cells in a medium containing DMEM/F12 (1:1, Sigma), insulin (25 μg/ml), transferrin (50 μg/ml), sodium selenite (30 nM), laminin (1 μg/ml, Sigma), progesterone (20 nM, Sigma), putrescin (100 μM, Sigma), penicillin (100 units/ml), streptomycin (100 μg/ml), EGF (20 ng/ml), bFGF (10 ng/ml), 2% B 27 supplement, nicotinamide (10 mM, Sigma). Indirect immunofluorescence analysis was carried out as described above using a primary antibody specific for insulin (Sigma).

### Example 6

Cultured Rat IS Cells Display Normal Karyotype.

Due to their rapid proliferation in culture established, embryonic stem cells often contain abnormal karyotypes (Abbondanzo, S. J. et al., Meth. Enzymol. 225: 803-823, 1993). However, stem cells exhibiting normal diploid karyotypes are preferred for clinical and other purposes. To determine whether IS-derived cells possess a normal karyotype after isolation and culture, rat IS cells which were cultured as described herein were tested by examining the cell's chromosomes for both structural and numerical abnormalities (FIG. 14). The karyotype of two independent clones (line 5 and line 30) from rat IS cells were analysed by, growing the cells on 6 cm petri dishes which were subsequently treated with 0.05 μg/ml colchicin for 90 min 2 days after the last subculture. Cells were detached with trypsin (0.2%) EDTA (0.02%) 1:1 and resuspended in the cell line-specific media. After centrifugation (1000 rpm, 5 min), the pellet was resuspended in hypotonic KCl solution (37° C.) by continuous shaking and incubated at 37° C. for 10 min. Cells were fixed twice with glacial acetic acid:methanol (1:3) and shaken continually during the fixation. The third fixation was done at 4° C. for 60 min. The cellular preparations were dropped on ice-cold microscopical slides and chromosomes were stained by orcein acetic acid (2%). Rat IS cells possessed normal chromosome number (n=42), and normal structural composition.

### Example 7

Distinct Nestin-positive Cells are Detected in the Mouse Intestinal Tissue

Immunohistochemistry revealed a significant expression of Cdx1 and a weak expression of Tcf4 (not shown) in the crypt and transit amplifying (TA) region of the intestinal epithelium of adult Rosa26 mice. Areas within the Paneth cell and the villus regions were not labelled by Cdx1j (FIG. 15A) or Tcf4. In addition, a few, single nestin-positive cells were found that were strongly labelled (see inserts, in FIG. 15B) and detected in the middle to upper part of the TA region. This finding of distinct nestin-positive cells in the intestinal epithelium prompted us to apply strategies which have been shown to be successful for the proliferation of nestin-positive cells (Lee et al., 2000, supra; Rolletschek et al., 2001, supra). Bouin-fixed, paraffin-embedded tissue specimens were sectioned at 5 μm. At least, two tissue blocks from different intestinal regions were examined per animal. Sections were mounted on silanized slides, deparaffinized in xylene and rehydrated through graded alcohols. Endogenous peroxidase was quenched by treatment with 3% hydrogen peroxide in methanol for 30 min. Non-specific binding of antibodies was blocked with 10% normal goat serum in PBS for 1 h at room temperature and incubated with the primary antisera overnight at 4° C. in a humidified chamber. All primary antibodies (mouse anti-Nestin antibody 1:10, rabbit anti-Cdx-1 antibody 1:800, mouse anti-TCF-4 antibody 1:500) were diluted in PBS with 1% BSA. Sections were rinsed off with PBST (phosphate-buffered saline Tween-20) and incubated with the respective multiple peroxidase labelled secondary antisera (DAKO EnVision™ and horse radish peroxidase (HRP)-goat anti-mouse IgG and HRP-goat anti-rabbit IgG). Antigen was visualized with diamino-benzidine (DAB) substrate. The enzyme reaction was stopped by immersion in water after 5 min. The specificity of immunostaining demonstrated by the absence of signal in sections incubated with control mouse IgG (DAKO) or in sections processed after omission of the primary antibody. Sections were then counterstained lightly with haematoxylin, dehydrated, cleared in xylene, mounted in DPX mounting medium (BDH Ldt, Pool, UK), and examined by conventional light microscopy.

### Example 8

Establishment of a Three-stage Protocol to Selectively Generate Nestin-positive Progenitor Cells in Vitro

The small intestine was prepared from 11 to 19 weeks old ROSA26 mice (Jackson Laboratory) constitutively expressing beta-gal in all cells. After flushing the lumenal content with Hanks balanced salt solution (HBSS, pH 7.3; Gibco BRL, Life Technologies, Eggenstein, Germany) containing penicillin (0.05 U/ml), streptomycin (0.05 μg/ml), gentamycin (50 μg/ml) and amphotericin (2.5 μg/ml; all from GIBCO), the tissue was cut longitudinally and pieces were washed (10×) in HBSS. Cells and aggregates of the intestinal epithelium or intestinal tissue were isolated by scraping and enzymatic dissociation with accutase 20-45-min (PAA Laboratories GmbH, Linz, Austria). By using similar techniques (Evans et al. (1992) J. Cell Sci., 101 219-231, Booth et al. (1999) Exp. Cell Res., 249, 359-36), functional intestinal stem cells have been isolated. These cell suspensions were cultured on feeder layer (FL) cells of mitomycin C (50 μ/ml, stock-solution 0.2 mg/ml; Serva, Heidelberg, Germany)-inactivated mouse embryonic fibroblasts (Wobus et al. 2002, Meth. Mol. Bio. 185:127-156) in DMEM (Gibco) with the following additives: 2 mM L-glutamine (stock solution 1:100, Gibco), 0,5 μmjM β-mercaptbethanol (β-ME, stock solution 1:100, Serva), non-essential amino acids (NEAA, stock solution 1:100, Gibco), 15% fetal calf serum (FCS, selected batches; Biochrom KG, Berlin, Germany), gentamycin (0.5 μg/ml) and amphotericin (0.25 μg/ml). The following cytokines and growth factors were used: LIF (10 ng/ml, for preparation see, bFGF (10 ng/ml) and EGF (20 ng/ml; both from Strathmann Biotech GmbH, Hannover, Germany). Both, bFGF and EGF are known to regulate the proliferation of intestinal epithelial cells (Houchen et al. (1999) Am. J. Phys. 276, G249-258, Potten et al. (1995) Gut, 36, 864-873). The growth factors were added every 2 days and all cultures were incubated at 37° C. and 5% CO₂. At day 4 or 5, IE-derived cells were dissociated with 0.2% trypsin (Gibco): 0.02% EDTA (Sigma) in PBS 1:1, and subcultured on fresh FL cells.

IE-derived cells were either continuously cultured on FL in DMEM+15% FCS containing LIF+bFGF for 8-14 passages followed by selective differentiation (Stage 1, see FIG. 16), or IE-derived clusters were generated by continuous feeder layer culture (2 to 3 subcultures) for 9 to 14 days (Stage 2, see FIG. 16). IE-derived clusters (Stage 2) were dissociated with collagenase (278 U/ml, Sigma, Steinheim, Germany): dispase (4 U/ml, Gibco) 1:1 at day 9 and transferred into bacteriological petri dishes as suspension culture in DMEM supplemented by additives (see above), 15% FCS, LIF (10 ng/ml), forskolin (10 μM; Sigma) and bFGF (10 ng/ml). After 5 days culture in suspension, IE-derived EB-like aggregates (Stage 3, see FIG. 16) were generated. For immunofluorescence analysis, these IE-derived EBs were plated onto gelatine-coated (0.1% in PBS, Fluka, Germany) 24 microwell-plates with coverslips and cultured 2 days in the same medium used for suspension culture (see above). For the generation of IE-EB-derived cell lines, EB-like aggregates were transferred into different media: Medium I is composed of EGM-2MV, heparin (Clonetics, Bio Whittaker, Verviers, Belgium) and LIF (10 ng/ml), Medium II is based on Iscove's modification of DMEM (IMDM; Gibco), supplemented by LIF (10 ng/ml), TGF β1 (2 ng/ml; Strathmann Biotech GmbH, Germany), bFGF (50 ng/ml), 20% FCS, gentamycin (0.5 μg/ml) and amphotericin (0.25 μg/ml). Medium III (used for single cell cloning) is composed of DMEM/F12 supplemented by 20 nM progesteron, 100 μM putrescin, 1 μg/ml laminin, 25 μg/ml insulin, 30 nM sodium selenite (all from Sigma), 50 μg/ml transferrin (Gibco) (=“B2”), 5% FCS, LIF, bFGF and EGF (for concentrations, see above).

Culture plates were coated with rat collagen I (0.05 mg/ml, Becton Dickinson, Heidelberg, Germany), gelatine (0.1%, Fluka) and poly-L-ornithine (0.1 mg/ml)/laminin (0.001 mg/ml) (Sigma), respectively. After separate cultivation of EB-like aggregates in single wells of 24 microwell-plates in medium I and II, IE-EB-derived cell lines were generated (see FIG. 16) that have been originally cultivated in DMEM without additional factors, or in DMEM+LIF, and DMEM+LIF+bFGF+EGF, respectively (for concentrations, see above).

The application of LIF and growth factors during the cultivation of IE-derived cells on embryonic fibroblast feeder layers, selectively supported the development of nestin-positive cells. The inventors noticed that the two different procedures (Stage 1 versus Stage 2 and 3) resulted in different cell populations with different properties. In one protocol generating Stage 1 cells, IE cells were continuously and selectively cultivated (for 8 to 14 passages) on feeder cells before induction of differentiation, whereas in the other protocol, compacted clusters (Stage 2) appeared on FL (FIG. 16A, B), which were then transferred into suspension culture and further cultivated as embryoid body (EB)-like aggregates (Stage 3, FIG. 16C, D). These compacted EB-like aggregates were then transferred onto different extracellular matrix proteins and propagated as EB-derived cell lines.

When intestinal epithelial cells of Rosa26 mice were cultivated according to these two procedures, nestin-positive cells were generated at all three stages. Because nestin-positive cells generated from ES cells have been shown to develop into neural (Lee et al., 2000, Nat. Biotechn. 18:675-679) and pancreatic (Lumelsky et al., 2001, supra) cells, similar protocols as being used for ES cells were applied to differentiate IE-derived cells into pancreatic and neural cell types. Additionally, hepatic and mesenchymal/mesodermal cell types were induced by specific protocols adapted from mouse ES cell differentiation studies.

### Example 9

IE-derived Clusters Propagated on Mouse Embryonic FL Co-express Alkaline Phosphatase and Nestin (Stage 2)

IE-derived clusters were analysed with respect to marker proteins which have been detected in undifferentiated ES cells, such as alkaline phosphatase activity (ALP). ALP was detected in IE-derived clusters after 14 (FIG. 17D, F, G) and 28 days cultivation on FL in growth medium with or without LIF or LIF+bFGF+EGF. Even single cells around the clusters were ALP-positive (FIG. 17G). No significant differences were observed in the ALP activity of IE-derived clusters after application of LIF (91%), LIF+bFGF+EGF (89%) and in control cultures (DMEM) without additional factors (85%, FIG. 17F), respectively, after 14 days of cultivation. After 28 days cultivation, the percentage of ALP-positive clusters decreased in all cultivation variants (data not shown), without significant differences between the variants. FL cells without IE showed no ALP-positive clusters (FIG. 17F).

In addition to ALP, most of the clusters were labelled by the intermediate filament protein nestin (FIG. 17 A,B and C,D). Both, neuroblasts (FIG. 17A) as well as uni- or bipolar nestin-positive cells (FIG. 17B,C) were found. The cells displayed a similar staining pattern as nestin-positive neural precursor cells developed from R1 ES cells (Rolletschek et al, 2001, supra). The size of these nestin-positive clusters and the number of nestin-positive cells increased with time of cultivation from day 9 to day 14 in all variants analysed, but most significantly, when cultivated in the presence of LIF, or LIF+bFGF+EGF, factors known to support the proliferation of nestin-positive cells (FIG. 17E).

### Example 10

IE-EB-like Aggregates Expressed Nestin and GFAP (Stage 3)

To investigate the properties of IE-EB-derived cells, immunocytochemical analysis of EB-derived clusters was performed. After plating of IE-EB-derived aggregates, uni- and bipolar nestin-positive neural precursor cell types (FIG. 18B) were found. The glial cell marker GFAP was detected in the IE-EB-derived outgrowths (FIG. 18C). Cdx1 was mainly expressed in the cytplasm of the cells (FIG. 18A). The results of the immunofluorescence analyses are summarized in Table 1. In control cultures (DMEM) without addition of LIF and growth factors during the primary culture (Stage 2), smaller EBs and fewer cells in the outgrowth with lower amounts of nestin (40%)- and GFAP (20%)- positive cells were found compared to cultures which were generated in the presence of LIF or LIF+bFGF+EGF (60% nestin- and 30% GFAP-positive cells, respectively). Cdx-1 was localized in the cytoplasm of 90% of the cells (FIG. 18A).

### Example 11

IE-derived Cells (Stage 1) Differentiated into Neural, but Mainly into Pancreatic and Hepatic Cells

IE-derived cells cultivated on FL were induced to differentiate into the neural, hepatic and pancreatic lineages. The IE-derived cells after hepatic differentiation induction in hepatocyte-promoting HCM medium for 14-28 days, were positive for nestin (between 40 to 80%), albumin (20-40%), α-1-antitrypsin (40-50%), and cytokeratin 18 (80%) (FIG. 19, Table 2). Further, differentiation of IE-derived cells into the pancreatic lineage according to a modified procedure of Lumelsky et al. for 14-28 days, revealed a high percentage of cells expressing insulin and glucagon in 40-70% of the cells (FIG. 19, Tab. 2). Nestin-positive cells were detected in the range of 50-80%. Addition of growth factors, such as LIF and bFGF into differentiation media did not significantly change the results. As evident from Tab. 4, the cells produced insulin at remarkable levels.

Insulin was detected intracellularly and at secreted levels. Neural differentiation by applying specific differentiation conditions revealed mainly nestin-positive cells in up to 70% of differentiated cells, whereas the differentiation into neuronal cells was only weak, and no glial cells were observed under the differentiation conditions used (see Tab. 2).

For differentiation into neural, pancreatic and hepatic phenotypes, IE-derived cells after cultivation on FL were dissociated (between passage number 8 to 14) by 0.2% trypsin (Gibco): 0.02% EDTA (Sigma) in PBS 1:1 and transferred to ECM-coated 24 microwell-plates with coverslips (as indicated).

For neural differentiation, IE-derived cells were plated onto poly-L-ornithine/laminin and cultured in DMEM/F12 supplemented by 20 nM progesteron, 100 μM putrescin, 1 μg/ml laminin, 25 μg/ml insulin, 30 nM sodium selenite (all from Sigma), 50 μg/ml transferrin (Gibco) (=“B2”) and 5% FCS for six days. The medium was changed every 2 days, and bFGF (10 ng/ml) and EGF (20 ng/ml) was added daily. After six days, the differentiation of neurons was induced by Neurobasal medium with 2% B27 (Gibco) and 10% FCS (see Rolletschek et al., 2001, supra).

For differentiation into pancreatic cell types, IE-derived cells were plated onto poly-L-ornithine/laminin and cultured in DMEM/F12 supplemented by “B2” components, 2% B27 and 10 mM niacinamide (Sigma) according to Lumelsky et al. 2001, supra, for 14 days.

For differentiation into hepatic cell types, IE-derived cells were plated onto rat collagen I and cultured in Hepatocyte Cultivation Medium HCM (Modified Williams E Medium, Clonetics, Cambrex Company, Belgium) with 20% FCS for 14 days, respectively.

### Example 12

IE-EB (Stage 3)—Derived Cell Lines Differentiated Efficiently into Neural and Mesenchymal/Mesodermal Cells

First, IE-EB-derived cell lines were generated by cultivation in media I and II (see Material and Methods) after plating onto different ECM proteins (see Tab. 3). Several IE-EB-derived cell lines originally cultivated in basic medium (DMEM) without additional factors (n=2), with LIF (n=3), and with LIF+bFGF+EGF, respectively, were generated (see morphology in FIG. 16E, F). The differentiation into neural, mesodermal/mesenchymal, pancreatic and hepatic cell types was induced by cultivation under specific differentiation conditions using protocols established for ES cells.

To investigate the differentiation capacity of IE-EB-derived cells into hepatic cell types, IE-EB-derived cells were cultivated in hepatocyte-promoting medium for about 14 days. Nestin was expressed in 80% of the cells. Dependent on the extracellular matrix proteins used, the number of IE-EB-derived cells expressing albumin varied between 10 (gelatine) and 70 (collagen I) % (see Table 3). α-1-antitrypsin (30-40%), cytokeratin 18 (80%) (FIG. 19, Tab. 2) were expressed after hepatic differentiation; cytokeratins 14 and 19 were not found. The highest percentage of expression of the investigated hepatic markers was observed in cultures derived from those cultivated on FL+LIF+bFGF+EGF.

Cultivation of IE-EB-derived cells in pancreatic differentiation medium (B2+B27+NA+5% FCS) for 19 days induced the formation of nestin-positive cells (80% of the cell population) and the differentiation of cells expressing insulin in up to 40% of the cells, when cultured on laminin-coated plates (Tab. 3), whereas gelatine did not support the formation of insulin-producing cells (see Tab. 3). IE-EB-derived cells were further cultivated under selective differentiation conditions promoting the differentiation into the neural lineage (EGM-2-MV and a differentiation medium according to Rolletschek et al. 2001, supra). As evident in Tab. 3 and FIG. 20, 70-80% of the cells were labelled by nestin (FIG. 20), and by neuronal markers, such as β-III-tubulin (up to 40%) and peripherin (up to 5%). Glial cells labelled by GFAP and oligodendrocytes-specific protein were labelled up to 60 and 5%, respectively (Tab. 3). There was a clear dependence of expression of neual proteins on the media. EGM2-MV and the neural differentiation medium supplemented by Neurobasal medium+B27 strongly supported the differentiation of neurons and glial cells. Culture variants with addition of LIF and LIF+bFGF+EGF to the primary cultures revealed a slightly higher amount of neural cells compared to the control (DMEM) group.

IE-EB-derived cells also showed an efficient differentiation of desmin (FIG. 20), and the cells stained also positive for the intermediate filament vimentin. The cells partially coexpressed desmin and nestin.

IE-derived EB-like aggregates were plated onto gelatine-, poly-L-ornithine/laminin (neural, pancreatic differentiation)- and collagen (hepatic differentiation)-coated 24 microwell-plates with coverslips and cultivated in DMEM supplemented by additives (see above), 15% FCS, LIF (10 ng/ml), forskolin (10 μM) and bFGF (10 ng/ml) for at least 8 days.

Neural differentiation was induced by medium change with DMEM/F12 supplemented by 20 nM progesteron, 100 μM putrescin, 1 μg/ml laminin, 25 μg/ml insulin, 30 nM sodium selenite, 50 μg/ml transferrin (=“B2”), 5% FCS, bFGF; EGF (for concentrations, see above) for 6 days, followed by differentiation in Neurobasal medium with 2% B27 supplement and 10% FCS (see above). For pancreatic and hepatic differentiation, IE-derived EBs were cultivated in DMEM/F12 supplemented by “B2” (see above), 2% B27, 10 mM niacinamide (see above) and 5% FCS according to Lumelsky et al., supra, and in Hepatocyte Cultivation Medium (HCM) with 20% FCS for 14 to 19 days, respectively. For differentiation into mesenchymal/mesodermal cells, EB-like aggregates were plated onto gelatine-coated coverslips and cultured in Medium II (see above) for 5 weeks.

### Example 13

Expression of SSEA-1 and Prominin in IE-derived Cells

The small intestine was prepared and cultured on feeder layer in similar way as described in Example 8. IE-derived cells cultured on feeder layer were analysed by immunofluorescence in primary cultures at day 2 and 5 and immunolabelled with SSEA-1-prominin-, Cdx-1- and nestin-specific antibodies (FIG. 21 D-O). SSEA-1 immunostaining was comparatively analysed to pluripotent R1 ES cells (FIG. 21A-C) [Nagy et al. (1993) Proc. Natl. Acad. Sci. USA 90, 8424-8428].

IE-derived and R1 cells were grown on microscopical slides. After rinsing with PBS, cultures were fixed with methanol: aceton (7:3) at −20° C. for 10 min. Goat serum (10%) or FCS (80%) in Tris-buffered saline Tween-20 (TBST; for all Cdx1 stainings) was used to inhibit unspecific labelling. Primary antibodies against the following markers were used at specific dilutions: nestin (Rat 401, 1:3), MC-480 (anti-SSEA-1, 1:10; both from Developmental Studies Hybridoma Bank, Iowa, USA), Cdx1 (1: 100, a kind gift of Dr. Barbara Meyer), prominin (mAb 13A4, 1:300, a kind gift of Drs. Denis Corbeil and Wieland Huttner). Antibody against Cdx1 and double stainings against Cdx1 and SSEA-1 (1:20) were incubated at 4° C. overnight, whereas all other antibodies and antibody combinations except anti-prominin were incubated at 37° C. for 60 min. For double stainings in combination with anti-prominin the following protocol according to and Corbeil et al., (2000) J. Biol. Chem., 275, 5512-5520, was used. Cells were washed with Ca/Mg-PBS, first at room temperature and then on ice, and labeled for 30 min at 4° C. by the addition of anti-prominin. Unbound antibodies were removed by five washes with ice-cold Ca/Mg-PBS containing 0.2% gelatin. For fixation, cells were covered with 3% paraformaldehyde at room temperature for 30 min or with methanol: aceton (7:3) for double staining with anti-SSEA-1 at −20° C. for 10 min. The fixative was removed by three washes with Ca/Mg-PBS, and the residual fixative was quenched for 30 min with 0.1 M glycine in PBS. Cells were then incubated with anti-nestin and anti-SSEA-1 antibodies as described. After rising (3×) in PBS, cells were then treated with appropriate fluorescence-labelled secondary antibodies. [SSEA-1 :Cy3 (mouse IgM; prominin: FITC ( rat IgG; Cdx1: FITC ( rabbit IgG; nestin: FITC ( mouse IgG; dilutions of Cyc3=1:700, FITC=1:100]. Hoechst 33342 (1 μg/ml) was used to label the nuclei. Specimens were analysed with the confocal laser scanning microscope (CLSM 410, Carl Zeiss, Jena, Germany) using following excitation lines/barrier filters: 364 nm/450-490BP (Hoechst 33342), 488 nm/510-525BP (FITC), 543 nm/570LP (Cy3).

The data (see FIG. 21) clearly show that pluripotent ES cells as well as specific subpopulations of IE-derived cells were labelled by the stage-specific embryonic cell surface antigen SSEA-1 characteristic for embryonic stem cells. Other subpopulations of IE-derived cells were labelled by SSEA-1 as well as prominin. Prominin is a 115 kDa membrane-associated glycoprotein known to be involved in asymmetric cell divisions and was characterised as orthologue of the human AC133 antigen (Corbeil et al., 2000, J. Biol. Chem. 275, 5512-5520)).

The data shown in FIG. 22 demonstrate that prominin-positive cells are also present after cultivation and differentiation into the pancreatic lineage. The prominin-positive cells are usually not co-labelled by a nestin-specific antibody. Thus, this invention shows that during cultivation on feeder layer (stage 1 cells) and during differentiation, IE-derived cells show expression of prominin suggesting that these cells have the capacity to self-renew and to differentiate.

1. A method of isolating and cultivating undifferentiated somatic intestinal stem or progenitor cells comprising:
(a) isolating intestinal stem or progenitor cells from an intestinal epithelium of mammalian original, and
(b) cultivating said intestinal stem cells on feeder cells under culture conditions allowing growth of said intestinal stem cells in an undifferentiated state.

2. The method of claim 1 further comprising:
(c) cultivating said intestinal stem cells to form embryoid bodies or embryoid-like bodies.

3. The method of claim 1 further comprising:
(d) cultivating said embryoid bodies or embryoid-like bodies under culture conditions allowing growth of said embryoid bodies or embryoid-like bodies in an undifferentiated state.

4. The method of claim 1, wherein said cultivating in step (b) and/or (d) comprises using culture media comprising factors and/or cells which inhibit cell differentiation.

5. The method of claim 1 further comprising:
(e) cultivating said intestinal stem or progenitor cells or said embryoid bodies or embryoid-like bodies under conditions allowing differentiation into specific cell types.

6. The method of claim 1 wherein the intestinal epithelium is of human origin.

7. The method of claim 1, wherein step (a) comprises:
(a1) obtaining epithelial tissue of mammalian origin from stomach, duodenum, jejunum, ileum, cecum, colon, rectum, and canal and/or appendix,
(a2) removing smooth muscle if present,
(a3) washing the remaining tissue in a buffered solution containing antibiotics, and
(a4) removing intestinal epithelial cells from the tissue by mechanical and/or enzymatic treatment.

8. The method of claim 1, wherein the feeder cells are selected from non-human or human mammalian embryonic cells, preferably embryonic fibroblastic cells, or human non-embryonic fibroblast cells.

9. The method of claim 4, wherein the factors are selected from
(i) cytokines and ligands of receptors capable of heterodimerisation with gp 130,
(ii) compounds which elevate intracellular camp levels,
(iii) mammalian growth factors and
(iv) any combinations thereof.

10. The method of claim 5, wherein step (e) comprises differentiation into cell types differing from intestinal cells

11. The method of claim 10, wherein the cell types are selected from neuronal cells, e.g. brain or spinal cord cells, pancreas cells, e.g. insulin-secreting pancreatic cell types, liver cells, heart cells, lung cells, kidney cells, muscle cells, skin cells, or hair cells.

12. The method of claim 1, wherein the intestinal stem cells are capable of expressing at least one marker for pluripotent cells.

13. The method of claim 12, wherein the marker for pluripotent cells is selected from Alkaline Phosphatase, SSEA-1, SSEA-3, SSEA-4, Oct-4, nestin, prominin/AC133, Cdx1, Tcf-4, vimentin, and any combination thereof.

14. The method of claim 1 further comprising:
(f) genetically modifying said intestinal stem cells or embryoid bodies or embryoid-like bodies.

15. The method of claim 14, wherein step (f) comprises: introducing a vector comprising a polynucleotide encompassing a polypeptide-coding region under control of a regulatory region into said intestinal stem cells or embryoid bodies or embryoid-like bodies.

16. The method of claim 14, wherein step (f) comprises: introducing a cell-type specific differentiation marker or a therapeutic polynucleotide into said intestinal stem cells or embryoid bodies or embryoid-like bodies.

17. The method of claim 1 further comprising:
(g) introducing an active ingredient, e.g. a pharmaceutically active ingredient, such as a protein, into said intestinal stem cells or embryoid bodies or embryoid-like bodies.

18. An isolated undifferentiated intestinal stem cell obtainable by the method of claim 1.

19. A culture of stem cells of claim 18.

20. The culture of claim 19, wherein the cells form embryoid or embryoid-like bodies.

21. The cell or cell culture of claim 18 capable of expressing at least one marker for pluripotent cells.

22. The cell or cell culture of claim 21, wherein the marker is selected from Alkaline Phosphatase, SSEA-1, SSEA-3, SSEA-4, Oct-4, nestin, prominin/AC133, Cdx1, Tcf-4, vimentin, and any combination thereof.

23. The cell or cell culture of claim 18 displaying a normal karyotype.

24. The cell or cell culture of claim 18 which is genetically modified.

25. The cell or cell culture of claim 18 comprising a heterologous active ingredient.

26. A differentiated cell derived from a cell or cell culture of claim 18.

27. A The differentiated cell of claim 26 which is selected from pancreas cells, neuronal cells, e.g. brain or spinal cord cells, liver cells, heart cells, lung cells, kidney cells, muscle cells, skin cells, or hair cells.

28. A culture of differentiated cells of claim 27.

29. A pharmaceutical composition comprising a cell or cell culture of claim 18.

30. The composition of claim 29 for therapeutic use.

31. The composition of claim 29 for tissue or cell repair.

32. The composition of claim 29 for the autologous treatment of diabetes.

33. The composition of claim 29 for the autologous treatment of cardiac diseases.

34. The composition of claim 29 for the autologous treatment of neuronal diseases.

35. The composition of claim 29 for transplantation.

36. Use of a cell or cell culture of claim 18 for the characterization of cellular responses to biologic, chemical or pharmacological agents.

37. The use of claim 36 in a drug-based screening assay.
